(19) United States
(12) Patent Application Publication
(10) Pub. No.: US 2013/0116336 A1
(43) Pub. Date: May 9, 2013

(54) TREATMENT OF ATAXIA TELANGIECTASIA

(71) Applicant: William D. Shrader, Belmont, CA (US)

(72) Inventor: William D. Shrader, Belmont, CA (US)

(21) Appl. No.: 13/639,494

(22) PCT Filed: Apr. 4, 2011
(86) PCT No.: PCT/US11/31133

§ 371 Date: Jan. 22, 2013

Related U.S. Application Data
(60) Provisional application No. 61/341,908, filed on Apr. 6, 2010.

(51) Int. Cl. A61K 31/122; C07C 50/28; A61K 45/06

(52) U.S. Cl. CPC A61K 31/122; A61K 45/06; C07C 50/28
USPC 514/689; 514/690; 568/377; 568/335

(57) ABSTRACT

The present invention relates to methods of treating Ataxia-Telangiectasia (A-T) or Ataxia-telangiectasia like disorder (ATLD) with compounds such as tocotrienol quinones and tocotrienol hydroquinones, including alpha-tocotrienol quinone, in order to alleviate symptoms of the disease.

### CROSS-REFERENCE TO RELATED APPLICATIONS

This application claims priority benefit of U.S. Provisional Patent Application No. 61/341,908, filed Apr. 6, 2010. The entire content of that application is hereby incorporated by reference herein.

### TECHNICAL FIELD OF THE INVENTION

The present invention relates to methods of treating Ataxia-Telangiectasia with compounds such as tocotrienol quinones and tocotrienol hydroquinones.

### BACKGROUND OF THE INVENTION

Ataxia-Telangiectasia (A-T), also known as Boder-Sedwig syndrome or Louis-Bar syndrome, is a rare neurodegenerative, inherited disease characterized by multiple devastating symptoms affecting different organs, such as immune dysfunction; cerebellar atrophy leading to neuromotor dysfunction and progressive ataxia; eye abnormalities including oculocutaneous telangiectasias; recurrent sinopulmonary infections; extreme sensitivity to ionizing radiation and chemically generated free radicals; and predisposition to cancer incidence. See Chun, Helen H. et al., “Ataxia-telangiectasia, and evolving phenotype,” DNA Repair, (2004), 3:1187-1196. A-T is caused by one or more mutations in the ATM gene (the gene abbreviation stands for “ataxia telangiectasia mutated”), which leads to lack of functional ATM, a protein kinase which is involved in cellular responses to DNA double strand breaks and possibly other oxidative stresses, as well as in regulation of several fundamental cellular functions (Andegeko, Y. et al. J. Biol. Chem. (2001), 276(41):38224-38230.)

A disease termed “Ataxia-telangiectasia like disorder” (ATLD) is also recognized as an extremely rare condition, which is characterized by progressive cerebellar ataxia, hypersensitivity to ionizing radiation and genomic instability (Hernandez, D. et al., J. Med. Genet. (1993), 30(2):135-40. ATLD has milder symptoms and shows slower progression. It can be distinguished from A-T by the absence of telangiectasias, and a later onset and slower progress of the disease. The gene mutation is hMre11 and maps to chromosome 11q21.

Ataxia-Telangiectasia (A-T) was first described and published in 1926 by the internist L. Syllaba and the neurologist K. Henner in Revue neurologique, (1926), 1: 541-560. They reported 3 adolescent Czech siblings with progressive choreoathetosis and ocular telangiectasia. In 1941, Denise Louis-Bar reported a case of a 9-year-old girl with progressive cerebellar ataxia, mental retardation and bilateral oculocutaneous telangiectasia (Confinia Neurologica, (1941), 4: 32-42. A-T was then referred to as the Louis-Bar syndrome. The clinical features and the familial incidence proposing an autosomal recessive mode of inheritance was introduced in 1958 by Boder E. & Sedgwick R P. “Ataxia-telangiectasia; a familial syndrome of progressive cerebellar ataxia, oculocutaneous telangiectasia and frequent pulmonary infection”; Pediatrics (1958), 21(4):526-54; and in 1988 Gatti, R. A. et al. identified the location of the specific gene responsible for A-T to be on chromosome 11q22-23 as referred to in Nature (1988) 336:577-580. The recognition of the gene mutation for A-T makes carrier detection and prenatal diagnosis now possible.

The incidence of Ataxia-Telangiectasia (A-T) is estimated at 1 in 40,000 to 1 in 100,000 live births worldwide and the ailment is progressive and generally fatal to patients by the time they reach their twenties. The cause of death of patients with A-T is attributed in more than 50% of cases to recurrent respiratory infections and in 30-50% of cases to cancer. Males and females are affected equally.

Patients with Ataxia-Telangiectasia (A-T) typically become affected with clinically apparent symptoms when a child begins to walk, and the ataxia affects motor skills, causing poor balance and slurred speech. Telangiectasia of the bulbar conjunctiva first appears at age 3-7 years, and subsequently involves the corners of the eyes or the surface of the ears and cheeks. Other features of this syndrome include retardation of growth, gonadal atrophy, dry coarse hair, and skin, premature graying of hair, vitiligo, and slower thinking speed after age 10 years. Many individuals with A-T have a weakened immune system with a defective T-lymphocyte system and a defective B-lymphocyte system associated with hypoplasia of the thymus and decreased levels of circulating immunoglobulin. Recurrent respiratory tract infections are common, frequently causing death in adolescence or young adulthood. Infections most commonly involve the lungs and sinuses, and are at least in part due to the immunodeficiency of A-T patients. Another factor that may contribute to lung infections is the swallowing dysfunction of A-T patients that results in solid food and liquid being aspired and entering the trachea instead of the esophagus.

Patients with A-T have an increased risk for developing malignancies, particularly cancers of the immune system, particularly leukemia and Hodgkin's lymphoma. It has been also reported that heterozygous carriers of the gene for ataxia-telangiectasia have an excess risk of cancer, particularly breast cancer in women. (Swift M. et al., New England J. of Med. (1987), 316:1289-1294).

Cells from patients with A-T are very sensitive to DNA damaging agents, such as ionizing radiation (Taylor, A M et al., Nature (1967), 114: 617-62). Cells from patients with A-T lack functional ATM activity and show defective double-strand break repair, defective cell cycle checkpoint control and radiation sensitivity. (Meek, D W Nature Review Cancer (2009), 9(10):714-723.) Since many of these characteristics are observed upon normal aging, A-T may be regarded as a premature aging syndrome. Other features of the disease may include diabetes mellitus.

Cells from patients with Ataxia-Telangiectasia (A-T) are in a state of continuous oxidative stress. Ambrose, M. et al., Hum. Mol. Gen. (2007), 16(18): 2154-2164 have demonstrated that overall mitochondrial respiration and oxidation rates are greatly compromised in frozen and fresh peripheral blood lymphocytes derived from A-T patients, and suggest that A-T is similar to other progressive neurological disorders that are characterized by oxidative stress and intrinsic mitochondrial dysfunction.

There is presently no treatment for Ataxia-Telangiectasia (A-T), but treatments with flavonoids common in diet, such as quercetin, kaempherol, apigenin, or luteolin, have been suggested as treatment for their chemopreventative effects in animal models and human epidemiological studies; see Ferguson L R. “Role of plant polyphenols in genomic stability” Mutat. Res. (2001), 475:89-111.) Other flavonoids that have been suggested for treatment are green tea flavonoids (epigallocatechin gallate) that exert anti-cardiovascular, anti-carcinogenic and anti-neurodegenerative effects (Mandel S et al., J. Neurochem. (2004), 88:1555-1569. Unfortunately, there is no evidence that these treatments are particularly effective. Supportive therapy is recommended to alleviate secondary symptoms, and physical and occupational therapists should be included to help prevent the development of stiffness of muscles and to maintain functionality. Speech therapists can also be used to help with enunciation and reduction of speech slur. Sinopulmonary infections may be managed during a hospital stay and intravenous treatment with antibiotics.

There is thus a critical and unmet need for effective treatments Ataxia-Telangiectasia (A-T) and Ataxia-telangiectasia like disorder (ATLD).

### SUMMARY OF THE INVENTION

In one embodiment, the invention provides methods of treating Ataxia-Telangiectasia (A-T) and Ataxia-telangiectasia like disorder (ATLD) with specific compounds.

In another embodiment, the invention provides methods of treating an individual suffering from Ataxia-Telangiectasia (A-T) or Ataxia-telangiectasia like disorder (ATLD) with tocotrienol quinones, comprising administering a therapeutically effective amount of one or more tocotrienol quinones to an individual suffering from Ataxia-Telangiectasia (A-T) or Ataxia-telangiectasia like disorder (ATLD). In another embodiment, the invention provides methods of treating an individual suffering from Ataxia-Telangiectasia (A-T) with alpha-tocotrienol quinone, comprising administering a therapeutically effective amount of alpha-tocotrienol quinone to an individual suffering from Ataxia-Telangiectasia (A-T). In another embodiment, the invention provides methods of treating an individual suffering from Ataxia-telangiectasia like disorder (ATLD) with alpha-tocotrienol quinone, comprising administering a therapeutically effective amount of alpha-tocotrienol quinone to an individual suffering from Ataxia-telangiectasia like disorder (ATLD). In another embodiment, the invention provides methods of treating an individual suffering from Ataxia-Telangiectasia (A-T) with beta-tocotrienol quinone, comprising administering a therapeutically effective amount of beta-tocotrienol quinone to an individual suffering from Ataxia-Telangiectasia (A-T). In another embodiment, the invention provides methods of treating an individual suffering from Ataxia-telangiectasia like disorder (ATLD) with beta-tocotrienol quinone, comprising administering a therapeutically effective amount of beta-tocotrienol quinone to an individual suffering from Ataxia-telangiectasia like disorder (ATLD). In another embodiment, the invention provides methods of treating an individual suffering from Ataxia-Telangiectasia (A-T) with gamma-tocotrienol quinone, comprising administering a therapeutically effective amount of gamma-tocotrienol quinone to an individual suffering from Ataxia-Telangiectasia (A-T). In another embodiment, the invention provides methods of treating an individual suffering from Ataxia-telangiectasia like disorder (ATLD) with gamma-tocotrienol quinone, comprising administering a therapeutically effective amount of gamma-tocotrienol quinone to an individual suffering from Ataxia-telangiectasia like disorder (ATLD). In another embodiment, the invention provides methods of treating an individual suffering from Ataxia-Telangiectasia (A-T) with delta-tocotrienol quinone, comprising administering a therapeutically effective amount of delta-tocotrienol quinone to an individual suffering from Ataxia-Telangiectasia (A-T). In another embodiment, the invention provides methods of treating an individual suffering from Ataxia-telangiectasia like disorder (ATLD) with delta-tocotrienol quinone, comprising administering a therapeutically effective amount of delta-tocotrienol quinone to an individual suffering from Ataxia-telangiectasia like disorder (ATLD).

In another embodiment, the invention provides methods of treating an individual suffering from Ataxia-Telangiectasia (A-T) or Ataxia-telangiectasia like disorder (ATLD) with tocotrienol hydroquinones, comprising administering a therapeutically effective amount of one or more tocotrienol hydroquinones to an individual suffering from Ataxia-Telangiectasia (A-T) and/or Ataxia-telangiectasia like disorder (ATLD). In another embodiment, the invention provides methods of treating an individual suffering from Ataxia-Telangiectasia (A-T) with alpha-tocotrienol hydroquinone, comprising administering a therapeutically effective amount of alpha-tocotrienol hydroquinone to an individual suffering from Ataxia-Telangiectasia (A-T). In another embodiment, the invention provides methods of treating an individual suffering from Ataxia-telangiectasia like disorder (ATLD) with alpha-tocotrienol hydroquinone, comprising administering a therapeutically effective amount of alpha-tocotrienol hydroquinone to an individual suffering from Ataxia-telangiectasia like disorder (ATLD). In another embodiment, the invention provides methods of treating an individual suffering from Ataxia-Telangiectasia (A-T) with beta-tocotrienol hydroquinone, comprising administering a therapeutically effective amount of beta-tocotrienol hydroquinone to an individual suffering from Ataxia-Telangiectasia (A-T). In another embodiment, the invention provides methods of treating an individual suffering from Ataxia-telangiectasia like disorder (ATLD) with beta-tocotrienol hydroquinone, comprising administering a therapeutically effective amount of beta-tocotrienol hydroquinone to an individual suffering from Ataxia-telangiectasia like disorder (ATLD). In another embodiment, the invention provides methods of treating an individual suffering from Ataxia-Telangiectasia (A-T) with gamma-tocotrienol hydroquinone, comprising administering a therapeutically effective amount of gamma-tocotrienol hydroquinone to an individual suffering from Ataxia-Telangiectasia (A-T). In another embodiment, the invention provides methods of treating an individual suffering from Ataxia-telangiectasia like disorder (ATLD) with gamma-tocotrienol hydroquinone, comprising administering a therapeutically effective amount of gamma-tocotrienol hydroquinone to an individual suffering from Ataxia-telangiectasia like disorder (ATLD). In another embodiment, the invention provides methods of treating an individual suffering from Ataxia-Telangiectasia (A-T) with delta-tocotrienol hydroquinone, comprising administering a therapeutically effective amount of delta-tocotrienol hydroquinone to an individual suffering from Ataxia-Telangiectasia (A-T). In another embodiment, the invention provides methods of treating an individual suffering from Ataxia-telangiectasia like disorder (ATLD) with delta-tocotrienol hydroquinone, comprising administering a therapeutically effective amount of delta-tocotrienol hydroquinone to an individual suffering from Ataxia-telangiectasia like disorder (ATLD).

In one embodiment, the pharmaceutical composition used in treating the individual comprises alpha-tocotrienol quinone, where the alpha-tocotrienol quinone comprises at least about 30% by weight of the tocotrienols and tocotrienol quinones present in the preparation. In another embodiment, the pharmaceutical composition used in treating the individual comprises alpha-tocotrienol quinone, where the alpha-tocotrienol quinone comprises at least about 40% by weight of the tocotrienols and tocotrienol quinones present in the preparation. In another embodiment, the pharmaceutical composition used in treating the individual comprises alpha-tocotrienol quinone, where the alpha-tocotrienol quinone comprises at least about 50% by weight of the tocotrienols and tocotrienol quinones present in the preparation. In another embodiment, the pharmaceutical composition used in treating the individual comprises alpha-tocotrienol quinone, where the alpha-tocotrienol quinone comprises at least about 60% by weight of the tocotrienols and tocotrienol quinones present in the preparation. In another embodiment, the pharmaceutical composition used in treating the individual comprises alpha-tocotrienol quinone, where the alpha-tocotrienol quinone comprises at least about 70% by weight of the tocotrienols and tocotrienol quinones present in the preparation. In another embodiment, the pharmaceutical composition used in treating the individual comprises alpha-tocotrienol quinone, where the alpha-tocotrienol quinone comprises at least about 75% by weight of the tocotrienols and tocotrienol quinones present in the preparation. In another embodiment, the pharmaceutical composition used in treating the individual comprises alpha-tocotrienol quinone, where the alpha-tocotrienol quinone comprises at least about 80% by weight of the tocotrienols and tocotrienol quinones present in the preparation. In another embodiment, the pharmaceutical composition used in treating the individual comprises alpha-tocotrienol quinone, where the alpha-tocotrienol quinone comprises at least about 90% by weight of the tocotrienols and tocotrienol quinones present in the preparation. In another embodiment, the pharmaceutical composition used in treating the individual comprises alpha-tocotrienol quinone, where the alpha-tocotrienol quinone comprises at least about 95% by weight of the tocotrienols and tocotrienol quinones present in the preparation. In another embodiment, the pharmaceutical composition used in treating the individual comprises alpha-tocotrienol quinone, where the alpha-tocotrienol quinone comprises at least about 98% by weight of the tocotrienols and tocotrienol quinones present in the preparation. In another embodiment, the pharmaceutical composition used in treating the individual comprises alpha-tocotrienol quinone, where the alpha-tocotrienol quinone comprises at least about 99% by weight of the tocotrienols and tocotrienol quinones present in the preparation.

In one embodiment, the pharmaceutical composition used in treating the individual comprises alpha-tocotrienol quinone, where the alpha-tocotrienol quinone comprises at least about 30% by weight of the tocotrienols, tocotrienol quinones, and tocotrienol hydroquinones present in the preparation. In another embodiment, the pharmaceutical composition used in treating the individual comprises alpha-tocotrienol quinone, where the alpha-tocotrienol quinone comprises at least about 40% by weight of the tocotrienols, tocotrienol quinones, and tocotrienol hydroquinones present in the preparation. In another embodiment, the pharmaceutical composition used in treating the individual comprises alpha-tocotrienol quinone, where the alpha-tocotrienol quinone comprises at least about 50% by weight of the tocotrienols, tocotrienol quinones, and tocotrienol hydroquinones present in the preparation. In another embodiment, the pharmaceutical composition used in treating the individual comprises alpha-tocotrienol quinone, where the alpha-tocotrienol quinone comprises at least about 60% by weight of the tocotrienols, tocotrienol quinones, and tocotrienol hydroquinones present in the preparation. In another embodiment, the pharmaceutical composition used in treating the individual comprises alpha-tocotrienol quinone, where the alpha-tocotrienol quinone comprises at least about 70% by weight of the tocotrienols, tocotrienol quinones, and tocotrienol hydroquinones present in the preparation. In another embodiment, the pharmaceutical composition used in treating the individual comprises alpha-tocotrienol quinone, where the alpha-tocotrienol quinone comprises at least about 75% by weight of the tocotrienols, tocotrienol quinones, and tocotrienol hydroquinones present in the preparation. In another embodiment, the pharmaceutical composition used in treating the individual comprises alpha-tocotrienol quinone, where the alpha-tocotrienol quinone comprises at least about 80% by weight of the tocotrienols, tocotrienol quinones, and tocotrienol hydroquinones present in the preparation. In another embodiment, the pharmaceutical composition used in treating the individual comprises alpha-tocotrienol quinone, where the alpha-tocotrienol quinone comprises at least about 90% by weight of the tocotrienols, tocotrienol quinones, and tocotrienol hydroquinones present in the preparation. In another embodiment, the pharmaceutical composition used in treating the individual comprises alpha-tocotrienol quinone, where the alpha-tocotrienol quinone comprises at least about 95% by weight of the tocotrienols, tocotrienol quinones, and tocotrienol hydroquinones present in the preparation. In another embodiment, the pharmaceutical composition used in treating the individual comprises alpha-tocotrienol quinone, where the alpha-tocotrienol quinone comprises at least about 98% by weight of the tocotrienols, tocotrienol quinones, and tocotrienol hydroquinones present in the preparation. In another embodiment, the pharmaceutical composition used in treating the individual comprises alpha-tocotrienol quinone, where the alpha-tocotrienol quinone comprises at least about 99% by weight of the tocotrienols, tocotrienol quinones, and tocotrienol hydroquinones present in the preparation.

In one embodiment, the pharmaceutical composition used in treating the individual comprises alpha-tocotrienol quinone, where the alpha-tocotrienol quinone comprises at least about 30% by weight of the material present in the preparation, excluding the weight of any added pharmaceutical carriers or excipients. In another embodiment, the pharmaceutical composition used in treating the individual comprises alpha-tocotrienol quinone, where the alpha-tocotrienol quinone comprises at least about 40% by weight of the material present in the preparation, excluding the weight of any added pharmaceutical carriers or excipients. In another embodiment, the pharmaceutical composition used in treating the individual comprises alpha-tocotrienol quinone, where the alpha-tocotrienol quinone comprises at least about 50% by weight of the material present in the preparation, excluding the weight of any added pharmaceutical carriers or excipients. In another embodiment, the pharmaceutical composition used in treating the individual comprises alpha-tocotrienol quinone, where the alpha-tocotrienol quinone comprises at least about 60% by weight of the material present in the preparation, excluding the weight of any added pharmaceutical carriers or excipients. In another embodiment, the pharmaceutical composition used in treating the individual comprises alpha-tocotrienol quinone, where the alpha-tocotrienol quinone comprises at least about 70% by weight of the material present in the preparation, excluding the weight of any added pharmaceutical carriers or excipients. In another embodiment, the pharmaceutical composition used in treating the individual comprises alpha-tocotrienol quinone, where the alpha-tocotrienol quinone comprises at least about 75% by weight of the material present in the preparation, excluding the weight of any added pharmaceutical carriers or excipients. In another embodiment, the pharmaceutical composition used in treating the individual comprises alpha-tocotrienol quinone, where the alpha-tocotrienol quinone comprises at least about 80% by weight of the material present in the preparation, excluding the weight of any added pharmaceutical carriers or excipients. In another embodiment, the pharmaceutical composition used in treating the individual comprises alpha-tocotrienol quinone, where the alpha-tocotrienol quinone comprises at least about 90% by weight of the material present in the preparation, excluding the weight of any added pharmaceutical carriers or excipients. In another embodiment, the pharmaceutical composition used in treating the individual comprises alpha-tocotrienol quinone, where the alpha-tocotrienol quinone comprises at least about 95% by weight of the material present in the preparation, excluding the weight of any added pharmaceutical carriers or excipients. In another embodiment, the pharmaceutical composition used in treating the individual comprises alpha-tocotrienol quinone, where the alpha-tocotrienol quinone comprises at least about 98% by weight of the material present in the preparation, excluding the weight of any added pharmaceutical carriers or excipients. In another embodiment, the pharmaceutical composition used in treating the individual comprises alpha-tocotrienol quinone, where the alpha-tocotrienol quinone comprises at least about 99% by weight of the material present in the preparation, excluding the weight of any added pharmaceutical carriers or excipients.

In one embodiment, the invention provides unit dosage formulations of between about 50 mg to 500 mg of alpha-tocotrienol quinone, where the alpha-tocotrienol quinone present in the formulation comprises at least about 30%, at least about 40%, at least about 50%, at least about 60%, at least about 70%, at least about 75%, at least about 80%, at least about 90%, at least about 95%, at least about 98%, or at least about 99% by weight of the tocotrienols and tocotrienol quinones present in the preparation.

In one embodiment, the invention provides unit dosage formulations of between about 50 mg to 500 mg of alpha-tocotrienol quinone, where the alpha-tocotrienol quinone present in the formulation comprises at least about 30%, at least about 40%, at least about 50%, at least about 60%, at least about 70%, at least about 75%, at least about 80%, at least about 90%, at least about 95%, at least about 98%, or at least about 99% of the material present in the preparation, excluding the weight of any added pharmaceutical carriers or excipients.

Any of the embodiments of the pharmaceutical compositions, pharmaceutical formulations and unit dosage formulations of alpha-tocotrienol quinone can be used to treat an individual suffering from Ataxia-Telangiectasia (A-T) or Ataxia-telangiectasia like disorder (ATLD), such as an individual with Ataxia-Telangiectasia.

In one embodiment, the pharmaceutical composition used in treating the individual comprises beta-tocotrienol quinone, where the beta-tocotrienol quinone comprises at least about 30% by weight of the tocotrienols and tocotrienol quinones present in the preparation. In another embodiment, the pharmaceutical composition used in treating the individual comprises beta-tocotrienol quinone, where the beta-tocotrienol quinone comprises at least about 40% by weight of the tocotrienols and tocotrienol quinones present in the preparation. In another embodiment, the pharmaceutical composition used in treating the individual comprises beta-tocotrienol quinone, where the beta-tocotrienol quinone comprises at least about 50% by weight of the tocotrienols and tocotrienol quinones present in the preparation. In another embodiment, the pharmaceutical composition used in treating the individual comprises beta-tocotrienol quinone, where the beta-tocotrienol quinone comprises at least about 60% by weight of the tocotrienols and tocotrienol quinones present in the preparation. In another embodiment, the pharmaceutical composition used in treating the individual comprises beta-tocotrienol quinone, where the beta-tocotrienol quinone comprises at least about 70% by weight of the tocotrienols and tocotrienol quinones present in the preparation. In another embodiment, the pharmaceutical composition used in treating the individual comprises beta-tocotrienol quinone, where the beta-tocotrienol quinone comprises at least about 75% by weight of the tocotrienols and tocotrienol quinones present in the preparation. In another embodiment, the pharmaceutical composition used in treating the individual comprises beta-tocotrienol quinone, where the beta-tocotrienol quinone comprises at least about 80% by weight of the tocotrienols and tocotrienol quinones present in the preparation. In another embodiment, the pharmaceutical composition used in treating the individual comprises beta-tocotrienol quinone, where the beta-tocotrienol quinone comprises at least about 90% by weight of the tocotrienols and tocotrienol quinones present in the preparation. In another embodiment, the pharmaceutical composition used in treating the individual comprises beta-tocotrienol quinone, where the beta-tocotrienol quinone comprises at least about 95% by weight of the tocotrienols and tocotrienol quinones present in the preparation. In another embodiment, the pharmaceutical composition used in treating the individual comprises beta-tocotrienol quinone, where the beta-tocotrienol quinone comprises at least about 98% by weight of the tocotrienols and tocotrienol quinones present in the preparation. In another embodiment, the pharmaceutical composition used in treating the individual comprises beta-tocotrienol quinone, where the beta-tocotrienol quinone comprises at least about 99% by weight of the tocotrienols and tocotrienol quinones present in the preparation.

In one embodiment, the pharmaceutical composition used in treating the individual comprises beta-tocotrienol quinone, where the beta-tocotrienol quinone comprises at least about 30% by weight of the tocotrienols, tocotrienol quinones, and tocotrienol hydroquinones present in the preparation. In another embodiment, the pharmaceutical composition used in treating the individual comprises beta-tocotrienol quinone, where the beta-tocotrienol quinone comprises at least about 40% by weight of the tocotrienols, tocotrienol quinones, and tocotrienol hydroquinones present in the preparation. In another embodiment, the pharmaceutical composition used in treating the individual comprises beta-tocotrienol quinone, where the beta-tocotrienol quinone comprises at least about 50% by weight of the tocotrienols, tocotrienol quinones, and tocotrienol hydroquinones present in the preparation. In another embodiment, the pharmaceutical composition used in treating the individual comprises beta-tocotrienol quinone, where the beta-tocotrienol quinone comprises at least about 60% by weight of the tocotrienols, tocotrienol quinones, and tocotrienol hydroquinones present in the preparation. In another embodiment, the pharmaceutical composition used in treating the individual comprises beta-tocotrienol quinone, where the beta-tocotrienol quinone comprises at least about 70% by weight of the tocotrienols, tocotrienol quinones, and tocotrienol hydroquinones present in the preparation. In another embodiment, the pharmaceutical composition used in treating the individual comprises beta-tocotrienol quinone, where the beta-tocotrienol quinone comprises at least about 75% by weight of the tocotrienols, tocotrienol quinones, and tocotrienol hydroquinones present in the preparation. In another embodiment, the pharmaceutical composition used in treating the individual comprises beta-tocotrienol quinone, where the beta-tocotrienol quinone comprises at least about 80% by weight of the tocotrienols, tocotrienol quinones, and tocotrienol hydroquinones present in the preparation. In another embodiment, the pharmaceutical composition used in treating the individual comprises beta-tocotrienol quinone, where the beta-tocotrienol quinone comprises at least about 90% by weight of the tocotrienols, tocotrienol quinones, and tocotrienol hydroquinones present in the preparation. In another embodiment, the pharmaceutical composition used in treating the individual comprises beta-tocotrienol quinone, where the beta-tocotrienol quinone comprises at least about 95% by weight of the tocotrienols, tocotrienol quinones, and tocotrienol hydroquinones present in the preparation. In another embodiment, the pharmaceutical composition used in treating the individual comprises beta-tocotrienol quinone, where the beta-tocotrienol quinone comprises at least about 98% by weight of the tocotrienols, tocotrienol quinones, and tocotrienol hydroquinones present in the preparation. In another embodiment, the pharmaceutical composition used in treating the individual comprises beta-tocotrienol quinone, where the beta-tocotrienol quinone comprises at least about 99% by weight of the tocotrienols, tocotrienol quinones, and tocotrienol hydroquinones present in the preparation.

In one embodiment, the pharmaceutical composition used in treating the individual comprises beta-tocotrienol quinone, where the beta-tocotrienol quinone comprises at least about 30% by weight of the material present in the preparation, excluding the weight of any added pharmaceutical carriers or excipients. In another embodiment, the pharmaceutical composition used in treating the individual comprises beta-tocotrienol quinone, where the beta-tocotrienol quinone comprises at least about 40% by weight of the material present in the preparation, excluding the weight of any added pharmaceutical carriers or excipients. In another embodiment, the pharmaceutical composition used in treating the individual comprises beta-tocotrienol quinone, where the beta-tocotrienol quinone comprises at least about 50% by weight of the material present in the preparation, excluding the weight of any added pharmaceutical carriers or excipients. In another embodiment, the pharmaceutical composition used in treating the individual comprises beta-tocotrienol quinone, where the beta-tocotrienol quinone comprises at least about 60% by weight of the material present in the preparation, excluding the weight of any added pharmaceutical carriers or excipients. In another embodiment, the pharmaceutical composition used in treating the individual comprises beta-tocotrienol quinone, where the beta-tocotrienol quinone comprises at least about 70% by weight of the material present in the preparation, excluding the weight of any added pharmaceutical carriers or excipients. In another embodiment, the pharmaceutical composition used in treating the individual comprises beta-tocotrienol quinone, where the beta-tocotrienol quinone comprises at least about 75% by weight of the material present in the preparation, excluding the weight of any added pharmaceutical carriers or excipients. In another embodiment, the pharmaceutical composition used in treating the individual comprises beta-tocotrienol quinone, where the beta-tocotrienol quinone comprises at least about 80% by weight of the material present in the preparation, excluding the weight of any added pharmaceutical carriers or excipients. In another embodiment, the pharmaceutical composition used in treating the individual comprises beta-tocotrienol quinone, where the beta-tocotrienol quinone comprises at least about 90% by weight of the material present in the preparation, excluding the weight of any added pharmaceutical carriers or excipients. In another embodiment, the pharmaceutical composition used in treating the individual comprises beta-tocotrienol quinone, where the beta-tocotrienol quinone comprises at least about 95% by weight of the material present in the preparation, excluding the weight of any added pharmaceutical carriers or excipients. In another embodiment, the pharmaceutical composition used in treating the individual comprises beta-tocotrienol quinone, where the beta-tocotrienol quinone comprises at least about 98% by weight of the material present in the preparation, excluding the weight of any added pharmaceutical carriers or excipients. In another embodiment, the pharmaceutical composition used in treating the individual comprises beta-tocotrienol quinone, where the beta-tocotrienol quinone comprises at least about 99% by weight of the material present in the preparation, excluding the weight of any added pharmaceutical carriers or excipients.

In one embodiment, the invention provides unit dosage formulations of between about 50 mg to 500 mg of beta-tocotrienol quinone, where the beta-tocotrienol quinone present in the formulation comprises at least about 30%, at least about 40%, at least about 50%, at least about 60%, at least about 70%, at least about 75%, at least about 80%, at least about 90%, at least about 95%, at least about 98%, or at least about 99% by weight of the tocotrienols and tocotrienol quinones present in the preparation.

In one embodiment, the invention provides unit dosage formulations of between about 50 mg to 500 mg of beta-tocotrienol quinone, where the beta-tocotrienol quinone present in the formulation comprises at least about 30%, at least about 40%, at least about 50%, at least about 60%, at least about 70%, at least about 75%, at least about 80%, at least about 90%, at least about 95%, at least about 98%, or at least about 99% of the material present in the preparation, excluding the weight of any added pharmaceutical carriers or excipients.

Any of the embodiments of the pharmaceutical compositions, pharmaceutical formulations and unit dosage formulations of beta-tocotrienol quinone can be used to treat an individual suffering from Ataxia-Telangiectasia (A-T) or Ataxia-telangiectasia like disorder (ATLD), such as an individual with Ataxia-Telangiectasia.

In one embodiment, the pharmaceutical composition used in treating the individual comprises gamma-tocotrienol quinone, where the gamma-tocotrienol quinone comprises at least about 30% by weight of the tocotrienols and tocotrienol quinones present in the preparation. In another embodiment, the pharmaceutical composition used in treating the individual comprises gamma-tocotrienol quinone, where the gamma-tocotrienol quinone comprises at least about 40% by weight of the tocotrienols and tocotrienol quinones present in the preparation. In another embodiment, the pharmaceutical composition used in treating the individual comprises gamma-tocotrienol quinone, where the gamma-tocotrienol quinone comprises at least about 50% by weight of the tocotrienols and tocotrienol quinones present in the preparation. In another embodiment, the pharmaceutical composition used in treating the individual comprises gamma-tocotrienol quinone, where the gamma-tocotrienol quinone comprises at least about 60% by weight of the tocotrienols and tocotrienol quinones present in the preparation. In another embodiment, the pharmaceutical composition used in treating the individual comprises gamma-tocotrienol quinone, where the gamma-tocotrienol quinone comprises at least about 70% by weight of the tocotrienols and tocotrienol quinones present in the preparation. In another embodiment, the pharmaceutical composition used in treating the individual comprises gamma-tocotrienol quinone, where the gamma-tocotrienol quinone comprises at least about 75% by weight of the tocotrienols and tocotrienol quinones present in the preparation. In another embodiment, the pharmaceutical composition used in treating the individual comprises gamma-tocotrienol quinone, where the gamma-tocotrienol quinone comprises at least about 80% by weight of the tocotrienols and tocotrienol quinones present in the preparation. In another embodiment, the pharmaceutical composition used in treating the individual comprises gamma-tocotrienol quinone, where the gamma-tocotrienol quinone comprises at least about 90% by weight of the tocotrienols and tocotrienol quinones present in the preparation. In another embodiment, the pharmaceutical composition used in treating the individual comprises gamma-tocotrienol quinone, where the gamma-tocotrienol quinone comprises at least about 95% by weight of the tocotrienols and tocotrienol quinones present in the preparation. In another embodiment, the pharmaceutical composition used in treating the individual comprises gamma-tocotrienol quinone, where the gamma-tocotrienol quinone comprises at least about 98% by weight of the tocotrienols and tocotrienol quinones present in the preparation. In another embodiment, the pharmaceutical composition used in treating the individual comprises gamma-tocotrienol quinone, where the gamma-tocotrienol quinone comprises at least about 99% by weight of the tocotrienols and tocotrienol quinones present in the preparation.

In one embodiment, the pharmaceutical composition used in treating the individual comprises gamma-tocotrienol quinone, where the gamma-tocotrienol quinone comprises at least about 30% by weight of the tocotrienols, tocotrienol quinones, and tocotrienol hydroquinones present in the preparation. In another embodiment, the pharmaceutical composition used in treating the individual comprises gamma-tocotrienol quinone, where the gamma-tocotrienol quinone comprises at least about 40% by weight of the tocotrienols, tocotrienol quinones, and tocotrienol hydroquinones present in the preparation. In another embodiment, the pharmaceutical composition used in treating the individual comprises gamma-tocotrienol quinone, where the gamma-tocotrienol quinone comprises at least about 50% by weight of the tocotrienols, tocotrienol quinones, and tocotrienol hydroquinones present in the preparation. In another embodiment, the pharmaceutical composition used in treating the individual comprises gamma-tocotrienol quinone, where the gamma-tocotrienol quinone comprises at least about 60% by weight of the tocotrienols, tocotrienol quinones, and tocotrienol hydroquinones present in the preparation. In another embodiment, the pharmaceutical composition used in treating the individual comprises gamma-tocotrienol quinone, where the gamma-tocotrienol quinone comprises at least about 70% by weight of the tocotrienols, tocotrienol quinones, and tocotrienol hydroquinones present in the preparation. In another embodiment, the pharmaceutical composition used in treating the individual comprises gamma-tocotrienol quinone, where the gamma-tocotrienol quinone comprises at least about 75% by weight of the tocotrienols, tocotrienol quinones, and tocotrienol hydroquinones present in the preparation. In another embodiment, the pharmaceutical composition used in treating the individual comprises gamma-tocotrienol quinone, where the gamma-tocotrienol quinone comprises at least about 80% by weight of the tocotrienols, tocotrienol quinones, and tocotrienol hydroquinones present in the preparation. In another embodiment, the pharmaceutical composition used in treating the individual comprises gamma-tocotrienol quinone, where the gamma-tocotrienol quinone comprises at least about 90% by weight of the tocotrienols, tocotrienol quinones, and tocotrienol hydroquinones present in the preparation. In another embodiment, the pharmaceutical composition used in treating the individual comprises gamma-tocotrienol quinone, where the gamma-tocotrienol quinone comprises at least about 95% by weight of the tocotrienols, tocotrienol quinones, and tocotrienol hydroquinones present in the preparation. In another embodiment, the pharmaceutical composition used in treating the individual comprises gamma-tocotrienol quinone, where the gamma-tocotrienol quinone comprises at least about 98% by weight of the tocotrienols, tocotrienol quinones, and tocotrienol hydroquinones present in the preparation. In another embodiment, the pharmaceutical composition used in treating the individual comprises gamma-tocotrienol quinone, where the gamma-tocotrienol quinone comprises at least about 99% by weight of the tocotrienols, tocotrienol quinones, and tocotrienol hydroquinones present in the preparation.

In one embodiment, the pharmaceutical composition used in treating the individual comprises gamma-tocotrienol quinone, where the gamma-tocotrienol quinone comprises at least about 30% by weight of the material present in the preparation, excluding the weight of any added pharmaceutical carriers or excipients. In another embodiment, the pharmaceutical composition used in treating the individual comprises gamma-tocotrienol quinone, where the gamma-tocotrienol quinone comprises at least about 40% by weight of the material present in the preparation, excluding the weight of any added pharmaceutical carriers or excipients. In another embodiment, the pharmaceutical composition used in treating the individual comprises gamma-tocotrienol quinone, where the gamma-tocotrienol quinone comprises at least about 50% by weight of the material present in the preparation, excluding the weight of any added pharmaceutical carriers or excipients. In another embodiment, the pharmaceutical composition used in treating the individual comprises gamma-tocotrienol quinone, where the gamma-tocotrienol quinone comprises at least about 60% by weight of the material present in the preparation, excluding the weight of any added pharmaceutical carriers or excipients. In another embodiment, the pharmaceutical composition used in treating the individual comprises gamma-tocotrienol quinone, where the gamma-tocotrienol quinone comprises at least about 70% by weight of the material present in the preparation, excluding the weight of any added pharmaceutical carriers or excipients. In another embodiment, the pharmaceutical composition used in treating the individual comprises gamma-tocotrienol quinone, where the gamma-tocotrienol quinone comprises at least about 75% by weight of the material present in the preparation, excluding the weight of any added pharmaceutical carriers or excipients. In another embodiment, the pharmaceutical composition used in treating the individual comprises gamma-tocotrienol quinone, where the gamma-tocotrienol quinone comprises at least about 80% by weight of the material present in the preparation, excluding the weight of any added pharmaceutical carriers or excipients. In another embodiment, the pharmaceutical composition used in treating the individual comprises gamma-tocotrienol quinone, where the gamma-tocotrienol quinone comprises at least about 90% by weight of the material present in the preparation, excluding the weight of any added pharmaceutical carriers or excipients. In another embodiment, the pharmaceutical composition used in treating the individual comprises gamma-tocotrienol quinone, where the gamma-tocotrienol quinone comprises at least about 95% by weight of the material present in the preparation, excluding the weight of any added pharmaceutical carriers or excipients. In another embodiment, the pharmaceutical composition used in treating the individual comprises gamma-tocotrienol quinone, where the gamma-tocotrienol quinone comprises at least about 98% by weight of the material present in the preparation, excluding the weight of any added pharmaceutical carriers or excipients. In another embodiment, the pharmaceutical composition used in treating the individual comprises gamma-tocotrienol quinone, where the gamma-tocotrienol quinone comprises at least about 99% by weight of the material present in the preparation, excluding the weight of any added pharmaceutical carriers or excipients.

In one embodiment, the invention provides unit dosage formulations of between about 50 mg to 500 mg of gamma-tocotrienol quinone, where the gamma-tocotrienol quinone present in the formulation comprises at least about 30%, at least about 40%, at least about 50%, at least about 60%, at least about 70%, at least about 75%, at least about 80%, at least about 90%, at least about 95%, at least about 98%, or at least about 99% by weight of the tocotrienols and tocotrienol quinones present in the preparation.

In one embodiment, the invention provides unit dosage formulations of between about 50 mg to 500 mg of gamma-tocotrienol quinone, where the gamma-tocotrienol quinone present in the formulation comprises at least about 30%, at least about 40%, at least about 50%, at least about 60%, at least about 70%, at least about 75%, at least about 80%, at least about 90%, at least about 95%, at least about 98%, or at least about 99% of the material present in the preparation, excluding the weight of any added pharmaceutical carriers or excipients.

Any of the embodiments of the pharmaceutical compositions, pharmaceutical formulations and unit dosage formulations of gamma-tocotrienol quinone can be used to treat an individual suffering from Ataxia-Telangiectasia (A-T) or Ataxia-telangiectasia like disorder (ATLD), such as an individual with Ataxia-Telangiectasia.

In one embodiment, the pharmaceutical composition used in treating the individual comprises delta-tocotrienol quinone, where the delta-tocotrienol quinone comprises at least about 30% by weight of the tocotrienols and tocotrienol quinones present in the preparation. In another embodiment, the pharmaceutical composition used in treating the individual comprises delta-tocotrienol quinone, where the delta-tocotrienol quinone comprises at least about 40% by weight of the tocotrienols and tocotrienol quinones present in the preparation. In another embodiment, the pharmaceutical composition used in treating the individual comprises delta-tocotrienol quinone, where the delta-tocotrienol quinone comprises at least about 50% by weight of the tocotrienols and tocotrienol quinones present in the preparation. In another embodiment, the pharmaceutical composition used in treating the individual comprises delta-tocotrienol quinone, where the delta-tocotrienol quinone comprises at least about 60% by weight of the tocotrienols and tocotrienol quinones present in the preparation. In another embodiment, the pharmaceutical composition used in treating the individual comprises delta-tocotrienol quinone, where the delta-tocotrienol quinone comprises at least about 70% by weight of the tocotrienols and tocotrienol quinones present in the preparation. In another embodiment, the pharmaceutical composition used in treating the individual comprises delta-tocotrienol quinone, where the delta-tocotrienol quinone comprises at least about 75% by weight of the tocotrienols and tocotrienol quinones present in the preparation. In another embodiment, the pharmaceutical composition used in treating the individual comprises delta-tocotrienol quinone, where the delta-tocotrienol quinone comprises at least about 80% by weight of the tocotrienols and tocotrienol quinones present in the preparation. In another embodiment, the pharmaceutical composition used in treating the individual comprises delta-tocotrienol quinone, where the delta-tocotrienol quinone comprises at least about 90% by weight of the tocotrienols and tocotrienol quinones present in the preparation. In another embodiment, the pharmaceutical composition used in treating the individual comprises delta-tocotrienol quinone, where the delta-tocotrienol quinone comprises at least about 95% by weight of the tocotrienols and tocotrienol quinones present in the preparation. In another embodiment, the pharmaceutical composition used in treating the individual comprises delta-tocotrienol quinone, where the delta-tocotrienol quinone comprises at least about 98% by weight of the tocotrienols and tocotrienol quinones present in the preparation. In another embodiment, the pharmaceutical composition used in treating the individual comprises delta-tocotrienol quinone, where the delta-tocotrienol quinone comprises at least about 99% by weight of the tocotrienols and tocotrienol quinones present in the preparation.

In one embodiment, the pharmaceutical composition used in treating the individual comprises delta-tocotrienol quinone, where the delta-tocotrienol quinone comprises at least about 30% by weight of the tocotrienols, tocotrienol quinones, and tocotrienol hydroquinones present in the preparation. In another embodiment, the pharmaceutical composition used in treating the individual comprises delta-tocotrienol quinone, where the delta-tocotrienol quinone comprises at least about 40% by weight of the tocotrienols, tocotrienol quinones, and tocotrienol hydroquinones present in the preparation. In another embodiment, the pharmaceutical composition used in treating the individual comprises delta-tocotrienol quinone, where the delta-tocotrienol quinone comprises at least about 50% by weight of the tocotrienols, tocotrienol quinones, and tocotrienol hydroquinones present in the preparation. In another embodiment, the pharmaceutical composition used in treating the individual comprises delta-tocotrienol quinone, where the delta-tocotrienol quinone comprises at least about 60% by weight of the tocotrienols, tocotrienol quinones, and tocotrienol hydroquinones present in the preparation. In another embodiment, the pharmaceutical composition used in treating the individual comprises delta-tocotrienol quinone, where the delta-tocotrienol quinone comprises at least about 70% by weight of the tocotrienols, tocotrienol quinones, and tocotrienol hydroquinones present in the preparation. In another embodiment, the pharmaceutical composition used in treating the individual comprises delta-tocotrienol quinone, where the delta-tocotrienol quinone comprises at least about 75% by weight of the tocotrienols, tocotrienol quinones, and tocotrienol hydroquinones present in the preparation. In another embodiment, the pharmaceutical composition used in treating the individual comprises delta-tocotrienol quinone, where the delta-tocotrienol quinone comprises at least about 80% by weight of the tocotrienols, tocotrienol quinones, and tocotrienol hydroquinones present in the preparation. In another embodiment, the pharmaceutical composition used in treating the individual comprises delta-tocotrienol quinone, where the delta-tocotrienol quinone comprises at least about 90% by weight of the tocotrienols, tocotrienol quinones, and tocotrienol hydroquinones present in the preparation. In another embodiment, the pharmaceutical composition used in treating the individual comprises delta-tocotrienol quinone, where the delta-tocotrienol quinone comprises at least about 95% by weight of the tocotrienols, tocotrienol quinones, and tocotrienol hydroquinones present in the preparation. In another embodiment, the pharmaceutical composition used in treating the individual comprises delta-tocotrienol quinone, where the delta-tocotrienol quinone comprises at least about 98% by weight of the tocotrienols, tocotrienol quinones, and tocotrienol hydroquinones present in the preparation. In another embodiment, the pharmaceutical composition used in treating the individual comprises delta-tocotrienol quinone, where the delta-tocotrienol quinone comprises at least about 99% by weight of the tocotrienols, tocotrienol quinones, and tocotrienol hydroquinones present in the preparation.

In one embodiment, the pharmaceutical composition used in treating the individual comprises delta-tocotrienol quinone, where the delta-tocotrienol quinone comprises at least about 30% by weight of the material present in the preparation, excluding the weight of any added pharmaceutical carriers or excipients. In another embodiment, the pharmaceutical composition used in treating the individual comprises delta-tocotrienol quinone, where the delta-tocotrienol quinone comprises at least about 40% by weight of the material present in the preparation, excluding the weight of any added pharmaceutical carriers or excipients. In another embodiment, the pharmaceutical composition used in treating the individual comprises delta-tocotrienol quinone, where the delta-tocotrienol quinone comprises at least about 50% by weight of the material present in the preparation, excluding the weight of any added pharmaceutical carriers or excipients. In another embodiment, the pharmaceutical composition used in treating the individual comprises delta-tocotrienol quinone, where the delta-tocotrienol quinone comprises at least about 60% by weight of the material present in the preparation, excluding the weight of any added pharmaceutical carriers or excipients. In another embodiment, the pharmaceutical composition used in treating the individual comprises delta-tocotrienol quinone, where the delta-tocotrienol quinone comprises at least about 70% by weight of the material present in the preparation, excluding the weight of any added pharmaceutical carriers or excipients. In another embodiment, the pharmaceutical composition used in treating the individual comprises delta-tocotrienol quinone, where the delta-tocotrienol quinone comprises at least about 75% by weight of the material present in the preparation, excluding the weight of any added pharmaceutical carriers or excipients. In another embodiment, the pharmaceutical composition used in treating the individual comprises delta-tocotrienol quinone, where the delta-tocotrienol quinone comprises at least about 80% by weight of the material present in the preparation, excluding the weight of any added pharmaceutical carriers or excipients. In another embodiment, the pharmaceutical composition used in treating the individual comprises delta-tocotrienol quinone, where the delta-tocotrienol quinone comprises at least about 90% by weight of the material present in the preparation, excluding the weight of any added pharmaceutical carriers or excipients. In another embodiment, the pharmaceutical composition used in treating the individual comprises delta-tocotrienol quinone, where the delta-tocotrienol quinone comprises at least about 95% by weight of the material present in the preparation, excluding the weight of any added pharmaceutical carriers or excipients. In another embodiment, the pharmaceutical composition used in treating the individual comprises delta-tocotrienol quinone, where the delta-tocotrienol quinone comprises at least about 98% by weight of the material present in the preparation, excluding the weight of any added pharmaceutical carriers or excipients. In another embodiment, the pharmaceutical composition used in treating the individual comprises delta-tocotrienol quinone, where the delta-tocotrienol quinone comprises at least about 99% by weight of the material present in the preparation, excluding the weight of any added pharmaceutical carriers or excipients.

In one embodiment, the invention provides unit dosage formulations of between about 50 mg to 500 mg of delta-tocotrienol quinone, where the delta-tocotrienol quinone present in the formulation comprises at least about 30%, at least about 40%, at least about 50%, at least about 60%, at least about 70%, at least about 75%, at least about 80%, at least about 90%, at least about 95%, at least about 98%, or at least about 99% by weight of the tocotrienols and tocotrienol quinones present in the preparation.

In one embodiment, the invention provides unit dosage formulations of between about 50 mg to 500 mg of delta-tocotrienol quinone, where the delta-tocotrienol quinone present in the formulation comprises at least about 30%, at least about 40%, at least about 50%, at least about 60%, at least about 70%, at least about 75%, at least about 80%, at least about 90%, at least about 95%, at least about 98%, or at least about 99% of the material present in the preparation, excluding the weight of any added pharmaceutical carriers or excipients.

Any of the embodiments of the pharmaceutical compositions, pharmaceutical formulations and unit dosage formulations of delta-tocotrienol quinone can be used to treat an individual suffering from Ataxia-Telangiectasia (A-T) or Ataxia-telangiectasia like disorder (ATLD), such as an individual with Ataxia-Telangiectasia.

In one embodiment, the pharmaceutical composition used in treating the individual comprises alpha-tocotrienol hydroquinone, where the alpha-tocotrienol hydroquinone comprises at least about 30% by weight of the tocotrienols and tocotrienol hydroquinones present in the preparation. In another embodiment, the pharmaceutical composition used in treating the individual comprises alpha-tocotrienol hydroquinone, where the alpha-tocotrienol hydroquinone comprises at least about 40% by weight of the tocotrienols and tocotrienol hydroquinones present in the preparation. In another embodiment, the pharmaceutical composition used in treating the individual comprises alpha-tocotrienol hydroquinone, where the alpha-tocotrienol hydroquinone comprises at least about 50% by weight of the tocotrienols and tocotrienol hydroquinones present in the preparation. In another embodiment, the pharmaceutical composition used in treating the individual comprises alpha-tocotrienol hydroquinone, where the alpha-tocotrienol hydroquinone comprises at least about 60% by weight of the tocotrienols and tocotrienol hydroquinones present in the preparation. In another embodiment, the pharmaceutical composition used in treating the individual comprises alpha-tocotrienol hydroquinone, where the alpha-tocotrienol hydroquinone comprises at least about 70% by weight of the tocotrienols and tocotrienol hydroquinones present in the preparation. In another embodiment, the pharmaceutical composition used in treating the individual comprises alpha-tocotrienol hydroquinone, where the alpha-tocotrienol hydroquinone comprises at least about 75% by weight of the tocotrienols and tocotrienol hydroquinones present in the preparation. In another embodiment, the pharmaceutical composition used in treating the individual comprises alpha-tocotrienol hydroquinone, where the alpha-tocotrienol hydroquinone comprises at least about 80% by weight of the tocotrienols and tocotrienol hydroquinones present in the preparation. In another embodiment, the pharmaceutical composition used in treating the individual comprises alpha-tocotrienol hydroquinone, where the alpha-tocotrienol hydroquinone comprises at least about 90% by weight of the tocotrienols and tocotrienol hydroquinones present in the preparation. In another embodiment, the pharmaceutical composition used in treating the individual comprises alpha-tocotrienol hydroquinone, where the alpha-tocotrienol hydroquinone comprises at least about 95% by weight of the tocotrienols and tocotrienol hydroquinones present in the preparation. In another embodiment, the pharmaceutical composition used in treating the individual comprises alpha-tocotrienol hydroquinone, where the alpha-tocotrienol hydroquinone comprises at least about 98% by weight of the tocotrienols and tocotrienol hydroquinones present in the preparation. In another embodiment, the pharmaceutical composition used in treating the individual comprises alpha-tocotrienol hydroquinone, where the alpha-tocotrienol hydroquinone comprises at least about 99% by weight of the tocotrienols and tocotrienol hydroquinones present in the preparation.

In one embodiment, the pharmaceutical composition used in treating the individual comprises alpha-tocotrienol hydroquinone, where the alpha-tocotrienol hydroquinone comprises at least about 30% by weight of the tocotrienols, tocotrienol quinones, and tocotrienol hydroquinones present in the preparation. In another embodiment, the pharmaceutical composition used in treating the individual comprises alpha-tocotrienol hydroquinone, where the alpha-tocotrienol hydroquinone comprises at least about 40% by weight of the tocotrienols, tocotrienol quinones, and tocotrienol hydroquinones present in the preparation. In another embodiment, the pharmaceutical composition used in treating the individual comprises alpha-tocotrienol hydroquinone, where the alpha-tocotrienol hydroquinone comprises at least about 50% by weight of the tocotrienols, tocotrienol quinones, and tocotrienol hydroquinones present in the preparation. In another embodiment, the pharmaceutical composition used in treating the individual comprises alpha-tocotrienol hydroquinone, where the alpha-tocotrienol hydroquinone comprises at least about 60% by weight of the tocotrienols, tocotrienol quinones, and tocotrienol hydroquinones present in the preparation. In another embodiment, the pharmaceutical composition used in treating the individual comprises alpha-tocotrienol hydroquinone, where the alpha-tocotrienol hydroquinone comprises at least about 70% by weight of the tocotrienols, tocotrienol quinones, and tocotrienol hydroquinones present in the preparation. In another embodiment, the pharmaceutical composition used in treating the individual comprises alpha-tocotrienol hydroquinone, where the alpha-tocotrienol hydroquinone comprises at least about 75% by weight of the tocotrienols, tocotrienol quinones, and tocotrienol hydroquinones present in the preparation. In another embodiment, the pharmaceutical composition used in treating the individual comprises alpha-tocotrienol hydroquinone, where the alpha-tocotrienol hydroquinone comprises at least about 80% by weight of the tocotrienols, tocotrienol quinones, and tocotrienol hydroquinones present in the preparation. In another embodiment, the pharmaceutical composition used in treating the individual comprises alpha-tocotrienol hydroquinone, where the alpha-tocotrienol hydroquinone comprises at least about 90% by weight of the tocotrienols, tocotrienol quinones, and tocotrienol hydroquinones present in the preparation. In another embodiment, the pharmaceutical composition used in treating the individual comprises alpha-tocotrienol hydroquinone, where the alpha-tocotrienol hydroquinone comprises at least about 95% by weight of the tocotrienols, tocotrienol quinones, and tocotrienol hydroquinones present in the preparation. In another embodiment, the pharmaceutical composition used in treating the individual comprises alpha-tocotrienol hydroquinone, where the alpha-tocotrienol hydroquinone comprises at least about 98% by weight of the tocotrienols, tocotrienol quinones, and tocotrienol hydroquinones present in the preparation. In another embodiment, the pharmaceutical composition used in treating the individual comprises alpha-tocotrienol hydroquinone, where the alpha-tocotrienol hydroquinone comprises at least about 99% by weight of the tocotrienols, tocotrienol quinones, and tocotrienol hydroquinones present in the preparation.

In one embodiment, the pharmaceutical composition used in treating the individual comprises alpha-tocotrienol hydroquinone, where the alpha-tocotrienol hydroquinone comprises at least about 30% by weight of the material present in the preparation, excluding the weight of any added pharmaceutical carriers or excipients. In another embodiment, the pharmaceutical composition used in treating the individual comprises alpha-tocotrienol hydroquinone, where the alpha-tocotrienol hydroquinone comprises at least about 40% by weight of the material present in the preparation, excluding the weight of any added pharmaceutical carriers or excipients. In another embodiment, the pharmaceutical composition used in treating the individual comprises alpha-tocotrienol hydroquinone, where the alpha-tocotrienol hydroquinone comprises at least about 50% by weight of the material present in the preparation, excluding the weight of any added pharmaceutical carriers or excipients. In another embodiment, the pharmaceutical composition used in treating the individual comprises alpha-tocotrienol hydroquinone, where the alpha-tocotrienol hydroquinone comprises at least about 60% by weight of the material present in the preparation, excluding the weight of any added pharmaceutical carriers or excipients. In another embodiment, the pharmaceutical composition used in treating the individual comprises alpha-tocotrienol hydroquinone, where the alpha-tocotrienol hydroquinone comprises at least about 70% by weight of the material present in the preparation, excluding the weight of any added pharmaceutical carriers or excipients. In another embodiment, the pharmaceutical composition used in treating the individual comprises alpha-tocotrienol hydroquinone, where the alpha-tocotrienol hydroquinone comprises at least about 75% by weight of the material present in the preparation, excluding the weight of any added pharmaceutical carriers or excipients. In another embodiment, the pharmaceutical composition used in treating the individual comprises alpha-tocotrienol hydroquinone, where the alpha-tocotrienol hydroquinone comprises at least about 80% by weight of the material present in the preparation, excluding the weight of any added pharmaceutical carriers or excipients. In another embodiment, the pharmaceutical composition used in treating the individual comprises alpha-tocotrienol hydroquinone, where the alpha-tocotrienol hydroquinone comprises at least about 90% by weight of the material present in the preparation, excluding the weight of any added pharmaceutical carriers or excipients. In another embodiment, the pharmaceutical composition used in treating the individual comprises alpha-tocotrienol hydroquinone, where the alpha-tocotrienol hydroquinone comprises at least about 95% by weight of the material present in the preparation, excluding the weight of any added pharmaceutical carriers or excipients. In another embodiment, the pharmaceutical composition used in treating the individual comprises alpha-tocotrienol hydroquinone, where the alpha-tocotrienol hydroquinone comprises at least about 98% by weight of the material present in the preparation, excluding the weight of any added pharmaceutical carriers or excipients. In another embodiment, the pharmaceutical composition used in treating the individual comprises alpha-tocotrienol hydroquinone, where the alpha-tocotrienol hydroquinone comprises at least about 99% by weight of the material present in the preparation, excluding the weight of any added pharmaceutical carriers or excipients.

In one embodiment, the invention provides unit dosage formulations of between about 50 mg to 500 mg of alpha-tocotrienol hydroquinone, where the alpha-tocotrienol hydroquinone present in the formulation comprises at least about 30%, at least about 40%, at least about 50%, at least about 60%, at least about 70%, at least about 75%, at least about 80%, at least about 90%, at least about 95%, at least about 98%, or at least about 99% by weight of the tocotrienols and tocotrienol hydroquinones present in the preparation.

In one embodiment, the invention provides unit dosage formulations of between about 50 mg to 500 mg of alpha-tocotrienol hydroquinone, where the alpha-tocotrienol hydroquinone present in the formulation comprises at least about 30%, at least about 40%, at least about 50%, at least about 60%, at least about 70%, at least about 75%, at least about 80%, at least about 90%, at least about 95%, at least about 98%, or at least about 99% of the material present in the preparation, excluding the weight of any added pharmaceutical carriers or excipients.

Any of the embodiments of the pharmaceutical compositions, pharmaceutical formulations and unit dosage formulations of alpha-tocotrienol hydroquinone can be used to treat an individual suffering from Ataxia-Telangiectasia (A-T) or Ataxia-telangiectasia like disorder (ATLD), such as an individual with Ataxia-Telangiectasia.

In one embodiment, the pharmaceutical composition used in treating the individual comprises beta-tocotrienol hydroquinone, where the beta-tocotrienol hydroquinone comprises at least about 30% by weight of the tocotrienols and tocotrienol hydroquinones present in the preparation. In another embodiment, the pharmaceutical composition used in treating the individual comprises beta-tocotrienol hydroquinone, where the beta-tocotrienol hydroquinone comprises at least about 40% by weight of the tocotrienols and tocotrienol hydroquinones present in the preparation. In another embodiment, the pharmaceutical composition used in treating the individual comprises beta-tocotrienol hydroquinone, where the beta-tocotrienol hydroquinone comprises at least about 50% by weight of the tocotrienols and tocotrienol hydroquinones present in the preparation. In another embodiment, the pharmaceutical composition used in treating the individual comprises beta-tocotrienol hydroquinone, where the beta-tocotrienol hydroquinone comprises at least about 60% by weight of the tocotrienols and tocotrienol hydroquinones present in the preparation. In another embodiment, the pharmaceutical composition used in treating the individual comprises beta-tocotrienol hydroquinone, where the beta-tocotrienol hydroquinone comprises at least about 70% by weight of the tocotrienols and tocotrienol hydroquinones present in the preparation. In another embodiment, the pharmaceutical composition used in treating the individual comprises beta-tocotrienol hydroquinone, where the beta-tocotrienol hydroquinone comprises at least about 75% by weight of the tocotrienols and tocotrienol hydroquinones present in the preparation. In another embodiment, the pharmaceutical composition used in treating the individual comprises beta-tocotrienol hydroquinone, where the beta-tocotrienol hydroquinone comprises at least about 80% by weight of the tocotrienols and tocotrienol hydroquinones present in the preparation. In another embodiment, the pharmaceutical composition used in treating the individual comprises beta-tocotrienol hydroquinone, where the beta-tocotrienol hydroquinone comprises at least about 90% by weight of the tocotrienols and tocotrienol hydroquinones present in the preparation. In another embodiment, the pharmaceutical composition used in treating the individual comprises beta-tocotrienol hydroquinone, where the beta-tocotrienol hydroquinone comprises at least about 95% by weight of the tocotrienols and tocotrienol hydroquinones present in the preparation. In another embodiment, the pharmaceutical composition used in treating the individual comprises beta-tocotrienol hydroquinone, where the beta-tocotrienol hydroquinone comprises at least about 98% by weight of the tocotrienols and tocotrienol hydroquinones present in the preparation. In another embodiment, the pharmaceutical composition used in treating the individual comprises beta-tocotrienol hydroquinone, where the beta-tocotrienol hydroquinone comprises at least about 99% by weight of the tocotrienols and tocotrienol hydroquinones present in the preparation.

In one embodiment, the pharmaceutical composition used in treating the individual comprises beta-tocotrienol hydroquinone, where the beta-tocotrienol hydroquinone comprises at least about 30% by weight of the tocotrienols, tocotrienol quinones, and tocotrienol hydroquinones present in the preparation. In another embodiment, the pharmaceutical composition used in treating the individual comprises beta-tocotrienol hydroquinone, where the beta-tocotrienol hydroquinone comprises at least about 40% by weight of the tocotrienols, tocotrienol quinones, and tocotrienol hydroquinones present in the preparation. In another embodiment, the pharmaceutical composition used in treating the individual comprises beta-tocotrienol hydroquinone, where the beta-tocotrienol hydroquinone comprises at least about 50% by weight of the tocotrienols, tocotrienol quinones, and tocotrienol hydroquinones present in the preparation. In another embodiment, the pharmaceutical composition used in treating the individual comprises beta-tocotrienol hydroquinone, where the beta-tocotrienol hydroquinone comprises at least about 60% by weight of the tocotrienols, tocotrienol quinones, and tocotrienol hydroquinones present in the preparation. In another embodiment, the pharmaceutical composition used in treating the individual comprises beta-tocotrienol hydroquinone, where the beta-tocotrienol hydroquinone comprises at least about 70% by weight of the tocotrienols, tocotrienol quinones, and tocotrienol hydroquinones present in the preparation. In another embodiment, the pharmaceutical composition used in treating the individual comprises beta-tocotrienol hydroquinone, where the beta-tocotrienol hydroquinone comprises at least about 75% by weight of the tocotrienols, tocotrienol quinones, and tocotrienol hydroquinones present in the preparation. In another embodiment, the pharmaceutical composition used in treating the individual comprises beta-tocotrienol hydroquinone, where the beta-tocotrienol hydroquinone comprises at least about 80% by weight of the tocotrienols, tocotrienol quinones, and tocotrienol hydroquinones present in the preparation. In another embodiment, the pharmaceutical composition used in treating the individual comprises beta-tocotrienol hydroquinone, where the beta-tocotrienol hydroquinone comprises at least about 90% by weight of the tocotrienols, tocotrienol quinones, and tocotrienol hydroquinones present in the preparation. In another embodiment, the pharmaceutical composition used in treating the individual comprises beta-tocotrienol hydroquinone, where the beta-tocotrienol hydroquinone comprises at least about 95% by weight of the tocotrienols, tocotrienol quinones, and tocotrienol hydroquinones present in the preparation. In another embodiment, the pharmaceutical composition used in treating the individual comprises beta-tocotrienol hydroquinone, where the beta-tocotrienol hydroquinone comprises at least about 98% by weight of the tocotrienols, tocotrienol quinones, and tocotrienol hydroquinones present in the preparation. In another embodiment, the pharmaceutical composition used in treating the individual comprises beta-tocotrienol hydroquinone, where the beta-tocotrienol hydroquinone comprises at least about 99% by weight of the tocotrienols, tocotrienol quinones, and tocotrienol hydroquinones present in the preparation.

In one embodiment, the pharmaceutical composition used in treating the individual comprises beta-tocotrienol hydroquinone, where the beta-tocotrienol hydroquinone comprises at least about 30% by weight of the material present in the preparation, excluding the weight of any added pharmaceutical carriers or excipients. In another embodiment, the pharmaceutical composition used in treating the individual comprises beta-tocotrienol hydroquinone, where the beta-tocotrienol hydroquinone comprises at least about 40% by weight of the material present in the preparation, excluding the weight of any added pharmaceutical carriers or excipients. In another embodiment, the pharmaceutical composition used in treating the individual comprises beta-tocotrienol hydroquinone, where the beta-tocotrienol hydroquinone comprises at least about 50% by weight of the material present in the preparation, excluding the weight of any added pharmaceutical carriers or excipients. In another embodiment, the pharmaceutical composition used in treating the individual comprises beta-tocotrienol hydroquinone, where the beta-tocotrienol hydroquinone comprises at least about 60% by weight of the material present in the preparation, excluding the weight of any added pharmaceutical carriers or excipients. In another embodiment, the pharmaceutical composition used in treating the individual comprises beta-tocotrienol hydroquinone, where the beta-tocotrienol hydroquinone comprises at least about 70% by weight of the material present in the preparation, excluding the weight of any added pharmaceutical carriers or excipients. In another embodiment, the pharmaceutical composition used in treating the individual comprises beta-tocotrienol hydroquinone, where the beta-tocotrienol hydroquinone comprises at least about 75% by weight of the material present in the preparation, excluding the weight of any added pharmaceutical carriers or excipients. In another embodiment, the pharmaceutical composition used in treating the individual comprises beta-tocotrienol hydroquinone, where the beta-tocotrienol hydroquinone comprises at least about 80% by weight of the material present in the preparation, excluding the weight of any added pharmaceutical carriers or excipients. In another embodiment, the pharmaceutical composition used in treating the individual comprises beta-tocotrienol hydroquinone, where the beta-tocotrienol hydroquinone comprises at least about 90% by weight of the material present in the preparation, excluding the weight of any added pharmaceutical carriers or excipients. In another embodiment, the pharmaceutical composition used in treating the individual comprises beta-tocotrienol hydroquinone, where the beta-tocotrienol hydroquinone comprises at least about 95% by weight of the material present in the preparation, excluding the weight of any added pharmaceutical carriers or excipients. In another embodiment, the pharmaceutical composition used in treating the individual comprises beta-tocotrienol hydroquinone, where the beta-tocotrienol hydroquinone comprises at least about 98% by weight of the material present in the preparation, excluding the weight of any added pharmaceutical carriers or excipients. In another embodiment, the pharmaceutical composition used in treating the individual comprises beta-tocotrienol hydroquinone, where the beta-tocotrienol hydroquinone comprises at least about 99% by weight of the material present in the preparation, excluding the weight of any added pharmaceutical carriers or excipients.

In one embodiment, the invention provides unit dosage formulations of between about 50 mg to 500 mg of beta-tocotrienol hydroquinone, where the beta-tocotrienol hydroquinone present in the formulation comprises at least about 30%, at least about 40%, at least about 50%, at least about 60%, at least about 70%, at least about 75%, at least about 80%, at least about 90%, at least about 95%, at least about 98%, or at least about 99% by weight of the tocotrienols and tocotrienol hydroquinones present in the preparation.

In one embodiment, the invention provides unit dosage formulations of between about 50 mg to 500 mg of beta-tocotrienol hydroquinone, where the beta-tocotrienol hydroquinone present in the formulation comprises at least about 30%, at least about 40%, at least about 50%, at least about 60%, at least about 70%, at least about 75%, at least about 80%, at least about 90%, at least about 95%, at least about 98%, or at least about 99% of the material present in the preparation, excluding the weight of any added pharmaceutical carriers or excipients.

Any of the embodiments of the pharmaceutical compositions, pharmaceutical formulations and unit dosage formulations of beta-tocotrienol hydroquinone can be used to treat an individual suffering from Ataxia-Telangiectasia (A-T) or Ataxia-telangiectasia like disorder (ATLD), such as an individual with Ataxia-Telangiectasia.

In one embodiment, the pharmaceutical composition used in treating the individual comprises gamma-tocotrienol hydroquinone, where the gamma-tocotrienol hydroquinone comprises at least about 30% by weight of the tocotrienols and tocotrienol hydroquinones present in the preparation. In another embodiment, the pharmaceutical composition used in treating the individual comprises gamma-tocotrienol hydroquinone, where the gamma-tocotrienol hydroquinone comprises at least about 40% by weight of the tocotrienols and tocotrienol hydroquinones present in the preparation. In another embodiment, the pharmaceutical composition used in treating the individual comprises gamma-tocotrienol hydroquinone, where the gamma-tocotrienol hydroquinone comprises at least about 50% by weight of the tocotrienols and tocotrienol hydroquinones present in the preparation. In another embodiment, the pharmaceutical composition used in treating the individual comprises gamma-tocotrienol hydroquinone, where the gamma-tocotrienol hydroquinone comprises at least about 60% by weight of the tocotrienols and tocotrienol hydroquinones present in the preparation. In another embodiment, the pharmaceutical composition used in treating the individual comprises gamma-tocotrienol hydroquinone, where the gamma-tocotrienol hydroquinone comprises at least about 70% by weight of the tocotrienols and tocotrienol hydroquinones present in the preparation. In another embodiment, the pharmaceutical composition used in treating the individual comprises gamma-tocotrienol hydroquinone, where the gamma-tocotrienol hydroquinone comprises at least about 75% by weight of the tocotrienols and tocotrienol hydroquinones present in the preparation. In another embodiment, the pharmaceutical composition used in treating the individual comprises gamma-tocotrienol hydroquinone, where the gamma-tocotrienol hydroquinone comprises at least about 80% by weight of the tocotrienols and tocotrienol hydroquinones present in the preparation. In another embodiment, the pharmaceutical composition used in treating the individual comprises gamma-tocotrienol hydroquinone, where the gamma-tocotrienol hydroquinone comprises at least about 90% by weight of the tocotrienols and tocotrienol hydroquinones present in the preparation. In another embodiment, the pharmaceutical composition used in treating the individual comprises gamma-tocotrienol hydroquinone, where the gamma-tocotrienol hydroquinone comprises at least about 95% by weight of the tocotrienols and tocotrienol hydroquinones present in the preparation. In another embodiment, the pharmaceutical composition used in treating the individual comprises gamma-tocotrienol hydroquinone, where the gamma-tocotrienol hydroquinone comprises at least about 98% by weight of the tocotrienols and tocotrienol hydroquinones present in the preparation. In another embodiment, the pharmaceutical composition used in treating the individual comprises gamma-tocotrienol hydroquinone, where the gamma-tocotrienol hydroquinone comprises at least about 99% by weight of the tocotrienols and tocotrienol hydroquinones present in the preparation.

In one embodiment, the pharmaceutical composition used in treating the individual comprises gamma-tocotrienol hydroquinone, where the gamma-tocotrienol hydroquinone comprises at least about 30% by weight of the tocotrienols, tocotrienol quinones, and tocotrienol hydroquinones present in the preparation. In another embodiment, the pharmaceutical composition used in treating the individual comprises gamma-tocotrienol hydroquinone, where the gamma-tocotrienol hydroquinone comprises at least about 40% by weight of the tocotrienols, tocotrienol quinones, and tocotrienol hydroquinones present in the preparation. In another embodiment, the pharmaceutical composition used in treating the individual comprises gamma-tocotrienol hydroquinone, where the gamma-tocotrienol hydroquinone comprises at least about 50% by weight of the tocotrienols, tocotrienol quinones, and tocotrienol hydroquinones present in the preparation. In another embodiment, the pharmaceutical composition used in treating the individual comprises gamma-tocotrienol hydroquinone, where the gamma-tocotrienol hydroquinone comprises at least about 60% by weight of the tocotrienols, tocotrienol quinones, and tocotrienol hydroquinones present in the preparation. In another embodiment, the pharmaceutical composition used in treating the individual comprises gamma-tocotrienol hydroquinone, where the gamma-tocotrienol hydroquinone comprises at least about 70% by weight of the tocotrienols, tocotrienol quinones, and tocotrienol hydroquinones present in the preparation. In another embodiment, the pharmaceutical composition used in treating the individual comprises gamma-tocotrienol hydroquinone, where the gamma-tocotrienol hydroquinone comprises at least about 75% by weight of the tocotrienols, tocotrienol quinones, and tocotrienol hydroquinones present in the preparation. In another embodiment, the pharmaceutical composition used in treating the individual comprises gamma-tocotrienol hydroquinone, where the gamma-tocotrienol hydroquinone comprises at least about 80% by weight of the tocotrienols, tocotrienol quinones, and tocotrienol hydroquinones present in the preparation. In another embodiment, the pharmaceutical composition used in treating the individual comprises gamma-tocotrienol hydroquinone, where the gamma-tocotrienol hydroquinone comprises at least about 90% by weight of the tocotrienols, tocotrienol quinones, and tocotrienol hydroquinones present in the preparation. In another embodiment, the pharmaceutical composition used in treating the individual comprises gamma-tocotrienol hydroquinone, where the gamma-tocotrienol hydroquinone comprises at least about 95% by weight of the tocotrienols, tocotrienol quinones, and tocotrienol hydroquinones present in the preparation. In another embodiment, the pharmaceutical composition used in treating the individual comprises gamma-tocotrienol hydroquinone, where the gamma-tocotrienol hydroquinone comprises at least about 98% by weight of the tocotrienols, tocotrienol quinones, and tocotrienol hydroquinones present in the preparation. In another embodiment, the pharmaceutical composition used in treating the individual comprises gamma-tocotrienol hydroquinone, where the gamma-tocotrienol hydroquinone comprises at least about 99% by weight of the tocotrienols, tocotrienol quinones, and tocotrienol hydroquinones present in the preparation.

In one embodiment, the pharmaceutical composition used in treating the individual comprises gamma-tocotrienol hydroquinone, where the gamma-tocotrienol hydroquinone comprises at least about 30% by weight of the material present in the preparation, excluding the weight of any added pharmaceutical carriers or excipients. In another embodiment, the pharmaceutical composition used in treating the individual comprises gamma-tocotrienol hydroquinone, where the gamma-tocotrienol hydroquinone comprises at least about 40% by weight of the material present in the preparation, excluding the weight of any added pharmaceutical carriers or excipients. In another embodiment, the pharmaceutical composition used in treating the individual comprises gamma-tocotrienol hydroquinone, where the gamma-tocotrienol hydroquinone comprises at least about 50% by weight of the material present in the preparation, excluding the weight of any added pharmaceutical carriers or excipients. In another embodiment, the pharmaceutical composition used in treating the individual comprises gamma-tocotrienol hydroquinone, where the gamma-tocotrienol hydroquinone comprises at least about 60% by weight of the material present in the preparation, excluding the weight of any added pharmaceutical carriers or excipients. In another embodiment, the pharmaceutical composition used in treating the individual comprises gamma-tocotrienol hydroquinone, where the gamma-tocotrienol hydroquinone comprises at least about 70% by weight of the material present in the preparation, excluding the weight of any added pharmaceutical carriers or excipients. In another embodiment, the pharmaceutical composition used in treating the individual comprises gamma-tocotrienol hydroquinone, where the gamma-tocotrienol hydroquinone comprises at least about 75% by weight of the material present in the preparation, excluding the weight of any added pharmaceutical carriers or excipients. In another embodiment, the pharmaceutical composition used in treating the individual comprises gamma-tocotrienol hydroquinone, where the gamma-tocotrienol hydroquinone comprises at least about 80% by weight of the material present in the preparation, excluding the weight of any added pharmaceutical carriers or excipients. In another embodiment, the pharmaceutical composition used in treating the individual comprises gamma-tocotrienol hydroquinone, where the gamma-tocotrienol hydroquinone comprises at least about 90% by weight of the material present in the preparation, excluding the weight of any added pharmaceutical carriers or excipients. In another embodiment, the pharmaceutical composition used in treating the individual comprises gamma-tocotrienol hydroquinone, where the gamma-tocotrienol hydroquinone comprises at least about 95% by weight of the material present in the preparation, excluding the weight of any added pharmaceutical carriers or excipients. In another embodiment, the pharmaceutical composition used in treating the individual comprises gamma-tocotrienol hydroquinone, where the gamma-tocotrienol hydroquinone comprises at least about 98% by weight of the material present in the preparation, excluding the weight of any added pharmaceutical carriers or excipients. In another embodiment, the pharmaceutical composition used in treating the individual comprises gamma-tocotrienol hydroquinone, where the gamma-tocotrienol hydroquinone comprises at least about 99% by weight of the material present in the preparation, excluding the weight of any added pharmaceutical carriers or excipients.

In one embodiment, the invention provides unit dosage formulations of between about 50 mg to 500 mg of gamma-tocotrienol hydroquinone, where the gamma-tocotrienol hydroquinone present in the formulation comprises at least about 30%, at least about 40%, at least about 50%, at least about 60%, at least about 70%, at least about 75%, at least about 80%, at least about 90%, at least about 95%, at least about 98%, or at least about 99% by weight of the tocotrienols and tocotrienol hydroquinones present in the preparation.

In one embodiment, the invention provides unit dosage formulations of between about 50 mg to 500 mg of gamma-tocotrienol hydroquinone, where the gamma-tocotrienol hydroquinone present in the formulation comprises at least about 30%, at least about 40%, at least about 50%, at least about 60%, at least about 70%, at least about 75%, at least about 80%, at least about 90%, at least about 95%, at least about 98%, or at least about 99% of the material present in the preparation, excluding the weight of any added pharmaceutical carriers or excipients.

Any of the embodiments of the pharmaceutical compositions, pharmaceutical formulations and unit dosage formulations of gamma-tocotrienol hydroquinone can be used to treat an individual suffering from Ataxia-Telangiectasia (A-T) or Ataxia-telangiectasia like disorder (ATLD), such as an individual with Ataxia-Telangiectasia.

In one embodiment, the pharmaceutical composition used in treating the individual comprises delta-tocotrienol hydroquinone, where the delta-tocotrienol hydroquinone comprises at least about 30% by weight of the tocotrienols and tocotrienol hydroquinones present in the preparation. In another embodiment, the pharmaceutical composition used in treating the individual comprises delta-tocotrienol hydroquinone, where the delta-tocotrienol hydroquinone comprises at least about 40% by weight of the tocotrienols and tocotrienol hydroquinones present in the preparation. In another embodiment, the pharmaceutical composition used in treating the individual comprises delta-tocotrienol hydroquinone, where the delta-tocotrienol hydroquinone comprises at least about 50% by weight of the tocotrienols and tocotrienol hydroquinones present in the preparation. In another embodiment, the pharmaceutical composition used in treating the individual comprises delta-tocotrienol hydroquinone, where the delta-tocotrienol hydroquinone comprises at least about 60% by weight of the tocotrienols and tocotrienol hydroquinones present in the preparation. In another embodiment, the pharmaceutical composition used in treating the individual comprises delta-tocotrienol hydroquinone, where the delta-tocotrienol hydroquinone comprises at least about 70% by weight of the tocotrienols and tocotrienol hydroquinones present in the preparation. In another embodiment, the pharmaceutical composition used in treating the individual comprises delta-tocotrienol hydroquinone, where the delta-tocotrienol hydroquinone comprises at least about 75% by weight of the tocotrienols and tocotrienol hydroquinones present in the preparation. In another embodiment, the pharmaceutical composition used in treating the individual comprises delta-tocotrienol hydroquinone, where the delta-tocotrienol hydroquinone comprises at least about 80% by weight of the tocotrienols and tocotrienol hydroquinones present in the preparation. In another embodiment, the pharmaceutical composition used in treating the individual comprises delta-tocotrienol hydroquinone, where the delta-tocotrienol hydroquinone comprises at least about 90% by weight of the tocotrienols and tocotrienol hydroquinones present in the preparation. In another embodiment, the pharmaceutical composition used in treating the individual comprises delta-tocotrienol hydroquinone, where the delta-tocotrienol hydroquinone comprises at least about 95% by weight of the tocotrienols and tocotrienol hydroquinones present in the preparation. In another embodiment, the pharmaceutical composition used in treating the individual comprises delta-tocotrienol hydroquinone, where the delta-tocotrienol hydroquinone comprises at least about 98% by weight of the tocotrienols and tocotrienol hydroquinones present in the preparation. In another embodiment, the pharmaceutical composition used in treating the individual comprises delta-tocotrienol hydroquinone, where the delta-tocotrienol hydroquinone comprises at least about 99% by weight of the tocotrienols and tocotrienol hydroquinones present in the preparation.

In one embodiment, the pharmaceutical composition used in treating the individual comprises delta-tocotrienol hydroquinone, where the delta-tocotrienol hydroquinone comprises at least about 30% by weight of the tocotrienols, tocotrienol quinones, and tocotrienol hydroquinones present in the preparation. In another embodiment, the pharmaceutical composition used in treating the individual comprises delta-tocotrienol hydroquinone, where the delta-tocotrienol hydroquinone comprises at least about 40% by weight of the tocotrienols, tocotrienol quinones, and tocotrienol hydroquinones present in the preparation. In another embodiment, the pharmaceutical composition used in treating the individual comprises delta-tocotrienol hydroquinone, where the delta-tocotrienol hydroquinone comprises at least about 50% by weight of the tocotrienols, tocotrienol quinones, and tocotrienol hydroquinones present in the preparation. In another embodiment, the pharmaceutical composition used in treating the individual comprises delta-tocotrienol hydroquinone, where the delta-tocotrienol hydroquinone comprises at least about 60% by weight of the tocotrienols, tocotrienol quinones, and tocotrienol hydroquinones present in the preparation. In another embodiment, the pharmaceutical composition used in treating the individual comprises delta-tocotrienol hydroquinone, where the delta-tocotrienol hydroquinone comprises at least about 70% by weight of the tocotrienols, tocotrienol quinones, and tocotrienol hydroquinones present in the preparation. In another embodiment, the pharmaceutical composition used in treating the individual comprises delta-tocotrienol hydroquinone, where the delta-tocotrienol hydroquinone comprises at least about 75% by weight of the tocotrienols, tocotrienol quinones, and tocotrienol hydroquinones present in the preparation. In another embodiment, the pharmaceutical composition used in treating the individual comprises delta-tocotrienol hydroquinone, where the delta-tocotrienol hydroquinone comprises at least about 80% by weight of the tocotrienols, tocotrienol quinones, and tocotrienol hydroquinones present in the preparation. In another embodiment, the pharmaceutical composition used in treating the individual comprises delta-tocotrienol hydroquinone, where the delta-tocotrienol hydroquinone comprises at least about 90% by weight of the tocotrienols, tocotrienol quinones, and tocotrienol hydroquinones present in the preparation. In another embodiment, the pharmaceutical composition used in treating the individual comprises delta-tocotrienol hydroquinone, where the delta-tocotrienol hydroquinone comprises at least about 95% by weight of the tocotrienols, tocotrienol quinones, and tocotrienol hydroquinones present in the preparation. In another embodiment, the pharmaceutical composition used in treating the individual comprises delta-tocotrienol hydroquinone, where the delta-tocotrienol hydroquinone comprises at least about 98% by weight of the tocotrienols, tocotrienol quinones, and tocotrienol hydroquinones present in the preparation. In another embodiment, the pharmaceutical composition used in treating the individual comprises delta-tocotrienol hydroquinone, where the delta-tocotrienol hydroquinone comprises at least about 99% by weight of the tocotrienols, tocotrienol quinones, and tocotrienol hydroquinones present in the preparation.

In one embodiment, the pharmaceutical composition used in treating the individual comprises delta-tocotrienol hydroquinone, where the delta-tocotrienol hydroquinone comprises at least about 30% by weight of the material present in the preparation, excluding the weight of any added pharmaceutical carriers or excipients. In another embodiment, the pharmaceutical composition used in treating the individual comprises delta-tocotrienol hydroquinone, where the delta-tocotrienol hydroquinone comprises at least about 40% by weight of the material present in the preparation, excluding the weight of any added pharmaceutical carriers or excipients. In another embodiment, the pharmaceutical composition used in treating the individual comprises delta-tocotrienol hydroquinone, where the delta-tocotrienol hydroquinone comprises at least about 50% by weight of the material present in the preparation, excluding the weight of any added pharmaceutical carriers or excipients. In another embodiment, the pharmaceutical composition used in treating the individual comprises delta-tocotrienol hydroquinone, where the delta-tocotrienol hydroquinone comprises at least about 60% by weight of the material present in the preparation, excluding the weight of any added pharmaceutical carriers or excipients. In another embodiment, the pharmaceutical composition used in treating the individual comprises delta-tocotrienol hydroquinone, where the delta-tocotrienol hydroquinone comprises at least about 70% by weight of the material present in the preparation, excluding the weight of any added pharmaceutical carriers or excipients. In another embodiment, the pharmaceutical composition used in treating the individual comprises delta-tocotrienol hydroquinone, where the delta-tocotrienol hydroquinone comprises at least about 75% by weight of the material present in the preparation, excluding the weight of any added pharmaceutical carriers or excipients. In another embodiment, the pharmaceutical composition used in treating the individual comprises delta-tocotrienol hydroquinone, where the delta-tocotrienol hydroquinone comprises at least about 80% by weight of the material present in the preparation, excluding the weight of any added pharmaceutical carriers or excipients. In another embodiment, the pharmaceutical composition used in treating the individual comprises delta-tocotrienol hydroquinone, where the delta-tocotrienol hydroquinone comprises at least about 90% by weight of the material present in the preparation, excluding the weight of any added pharmaceutical carriers or excipients. In another embodiment, the pharmaceutical composition used in treating the individual comprises delta-tocotrienol hydroquinone, where the delta-tocotrienol hydroquinone comprises at least about 95% by weight of the material present in the preparation, excluding the weight of any added pharmaceutical carriers or excipients. In another embodiment, the pharmaceutical composition used in treating the individual comprises delta-tocotrienol hydroquinone, where the delta-tocotrienol hydroquinone comprises at least about 98% by weight of the material present in the preparation, excluding the weight of any added pharmaceutical carriers or excipients. In another embodiment, the pharmaceutical composition used in treating the individual comprises delta-tocotrienol hydroquinone, where the delta-tocotrienol hydroquinone comprises at least about 99% by weight of the material present in the preparation, excluding the weight of any added pharmaceutical carriers or excipients.

In one embodiment, the invention provides unit dosage formulations of between about 50 mg to 500 mg of delta-tocotrienol hydroquinone, where the delta-tocotrienol hydroquinone present in the formulation comprises at least about 30%, at least about 40%, at least about 50%, at least about 60%, at least about 70%, at least about 75%, at least about 80%, at least about 90%, at least about 95%, at least about 98%, or at least about 99% by weight of the tocotrienols and tocotrienol hydroquinones present in the preparation.

In one embodiment, the invention provides unit dosage formulations of between about 50 mg to 500 mg of delta-tocotrienol hydroquinone, where the delta-tocotrienol hydroquinone present in the formulation comprises at least about 30%, at least about 40%, at least about 50%, at least about 60%, at least about 70%, at least about 75%, at least about 80%, at least about 90%, at least about 95%, at least about 98%, or at least about 99% of the material present in the preparation, excluding the weight of any added pharmaceutical carriers or excipients.

Any of the embodiments of the pharmaceutical compositions, pharmaceutical formulations and unit dosage formulations of delta-tocotrienol hydroquinone can be used to treat an individual suffering from Ataxia-Telangiectasia (A-T) or Ataxia-telangiectasia like disorder (ATLD), such as an individual with Ataxia-Telangiectasia.

In another embodiment, the invention provides methods of treating an individual suffering from Ataxia-Telangiectasia (A-T) or Ataxia-telangiectasia like disorder (ATLD) with compounds of Formula I, comprising administering a therapeutically effective amount of one or more compounds of Formula I to an individual suffering from Ataxia-Telangiectasia (A-T) and/or Ataxia-telangiectasia like disorder (ATLD):

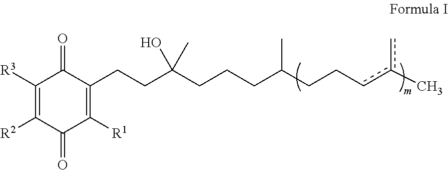

wherein the bonds indicated by a dashed line can be double or single, with the proviso that they are not both double within the same unit; and further proviso that at least one bond is a double bond;

R¹, R², and R³ are independently of each other hydrogen, (C₁-C₆) alkyl, or (C₁-C₆) alkoxy; and m is an integer from 0 to 12 inclusive, wherein each unit can be the same or different;

or any stereoisomer, mixture of stereoisomers, prodrug, metabolite, salt, crystalline form, non-crystalline form, hydrate or solvate thereof. In one embodiment, m is an integer from 1 to 12 inclusive.

In another embodiment, the invention provides methods of treating an individual suffering from Ataxia-Telangiectasia (A-T) or Ataxia-telangiectasia like disorder (ATLD) with compounds of Formula I-red, comprising administering a therapeutically effective amount of one or more compounds of Formula I-red to an individual suffering from Ataxia-Telangiectasia (A-T) and/or Ataxia-telangiectasia like disorder (ATLD), where the compounds of Formula I-red are the reduced (hydroquinone, that is, benzenediol) analogs of the compounds of Formula I.

In one embodiment, the individual suffering from A-T has a mutation, or at least one mutation, in the ATM gene located on chromosome 11q22-23. In another embodiment, the individual suffering from Ataxia-Telangiectasia (A-T) lacks ATM protein or ATM kinase activity, or lacks substantial amounts of ATM protein or ATM kinase activity, or has an amount of ATM protein or ATM kinase activity that is about 50% or lower than that of a healthy individual (an individual who does not suffer from A-T), or has an amount of ATM protein or ATM kinase activity that is about 20% or lower than that of a healthy individual (an individual who does not suffer from A-T), or has an amount of ATM protein or ATM kinase activity that is about 10% or lower than that of a healthy individual (an individual who does not suffer from A-T), or has an amount of ATM protein or ATM kinase activity that is about 5% or lower than that of a healthy individual (an individual who does not suffer from A-T), or has an amount of ATM protein or ATM kinase activity that is about 1% or lower than that of a healthy individual (an individual who does not suffer from A-T). In another embodiment, the individual suffering from A-T has a survival fraction of lymphoblastoid cell lines, established from a sample from the individual, and measured by colony survival assay (CSA) following exposure to a 1 Gray dose of gamma radiation, below about 21%. In another embodiment, the individual suffering from Ataxia-telangiectasia like disorder (ATLD) has a mutation, or has at least one mutation, in the hMre11 gene located on chromosome 11q21.

In one embodiment, the individual suffering from Ataxia-Telangiectasia (A-T) or Ataxia-telangiectasia like disorder (ATLD), such as an individual suffering from A-T, has one or more symptoms selected from the group consisting of: truncal ataxia (loss of control of body posture and body movement); peripheral ataxia (abnormal coordination of limbs); cerebellar ataxia; chorea (small jerks of the hands and feet which look like fidgeting); atheosis (slower twisting movements of the upper body); dystonia (adoption of stiff and twisted postures); myoclonic jerks (occasional uncontrolled jerks); difficulty in swallowing; tremors (shaking episodes of a limb which are like shivering); dysarthria (slurring of speech); vertical and horizontal sacchadic apraxia (restricted eye movements); telangiectasias (prominent blood vessels in the whites of the eyes or in the facial skin); immunodeficiency symptoms such as sinopulmonary infections (repeated colds and runny noses); cancer or cancerous tumors including lymphomas, leukemia and breast cancer; increased sensitivity to ionizing radiation (X rays and gamma rays); thymic hypoplasia; hypogonadism; genomic instability; and premature aging symptoms such as diabetes mellitus and progeria (hair graying, hair loss, wrinkling of the skin, bone deterioration, premature cataracts and need for reading glasses in children).

In one embodiment, the individual suffering from Ataxia-Telangiectasia (A-T) or Ataxia-telangiectasia like disorder (ATLD), such as an individual suffering from A-T, has one or more symptoms selected from the group consisting of ataxia; telangiectasia; sinopulmonary infections; cancer or cancerous tumors including lymphomas, leukemia and breast cancer; genomic instability; and sensitivity to ionizing radiation such as X rays or gamma rays.

In one embodiment, including any of the foregoing embodiments, the individual suffering from Ataxia-Telangiectasia (A-T) or Ataxia-telangiectasia like disorder (ATLD), such as an individual suffering from A-T, develops or has cancer or cancerous tumors. In one embodiment, the individual suffering from Ataxia-Telangiectasia (A-T) or Ataxia-telangiectasia like disorder (ATLD), such as an individual suffering from A-T, develops or has leukemia. In one embodiment, the individual suffering from Ataxia-Telangiectasia (A-T) or Ataxia-telangiectasia like disorder (ATLD), such as an individual suffering from A-T, develops or has lymphomas. In one embodiment, the individual suffering from Ataxia-Telangiectasia (A-T) or Ataxia-telangiectasia like disorder (ATLD), such as an individual suffering from A-T, develops or has breast cancer.

In one embodiment, where a therapeutically effective amount of one or more of alpha-tocotrienol quinone, alpha-tocotrienol hydroquinone, beta-tocotrienol quinone, beta-tocotrienol hydroquinone, gamma-tocotrienol quinone, gamma-tocotrienol hydroquinone, delta-tocotrienol quinone, or delta-tocotrienol hydroquinone, such as a therapeutically effective amount of alpha-tocotrienol quinone, is administered to an individual suffering from Ataxia-Telangiectasia (A-T) or Ataxia-telangiectasia like disorder (ATLD), such as an individual suffering from A-T, the individual has one or more symptoms selected from the group consisting of truncal ataxia (loss of control of body posture and body movement); peripheral ataxia (abnormal coordination of limbs); cerebellar ataxia; chorea (small jerks of the hands and feet which look like fidgeting); atheosis (slower twisting movements of the upper body); dystonia (adoption of stiff and twisted postures); myoclonic jerks (occasional uncontrolled jerks); swallowing dysfunction; tremors (shaking episodes of a limb which are like shivering); dysarthria (slurring of speech); vertical and horizontal sacchadic apraxia (restricted eye movements); telangiectasias (prominent blood vessels in the whites of the eyes or in the facial skin); immunodeficiency symptoms such as sinopulmonary infections (repeated colds and runny noses); cancer or cancerous tumors including lymphomas, leukemia and breast cancer; increased sensitivity to ionizing radiation (X rays and gamma rays); thymic hypoplasia; hypogonadism; genomic instability; and premature aging symptoms such as diabetes mellitus and progeria (hair graying, hair loss, wrinkling of the skin, bone deterioration, premature cataracts, and need for reading glasses in children).

In one embodiment, administration of a therapeutically effective amount of one or more of alpha-tocotrienol quinone, alpha-tocotrienol hydroquinone, beta-tocotrienol quinone, beta-tocotrienol hydroquinone, gamma-tocotrienol quinone, gamma-tocotrienol hydroquinone, delta-tocotrienol quinone, delta-tocotrienol hydroquinone, or any combination of two or more of the foregoing compounds, such as a therapeutically effective amount of alpha-tocotrienol quinone, to an individual suffering from Ataxia-Telangiectasia (A-T) or Ataxia-telangiectasia like disorder (ATLD), such as an individual suffering from A-T, alleviates, arrests the progression of, or reverses the occurrence of, one or more symptoms selected from the group consisting of ataxia, telangiectasia, sinopulmonary infections, lymphomas, leukemia, breast cancer, genomic instability, and sensitivity to ionizing radiation such as X rays or gamma rays. In another embodiment, administration of a therapeutically effective amount of alpha-tocotrienol quinone, alpha-tocotrienol hydroquinone, beta-tocotrienol quinone, beta-tocotrienol hydroquinone, gamma-tocotrienol quinone, gamma-tocotrienol hydroquinone, delta-tocotrienol quinone, delta-tocotrienol hydroquinone, or any combination of two or more of the foregoing compounds, such as a therapeutically effective amount of alpha-tocotrienol quinone, to an individual suffering from Ataxia-Telangiectasia (A-T), alleviates, arrests, or reverses the progression of, or prevents the occurrence of, ataxia. In another embodiment, administration of a therapeutically effective amount of alpha-tocotrienol quinone, alpha-tocotrienol hydroquinone, beta-tocotrienol quinone, beta-tocotrienol hydroquinone, gamma-tocotrienol quinone, gamma-tocotrienol hydroquinone, delta-tocotrienol quinone, delta-tocotrienol hydroquinone, or any combination of two or more of the foregoing compounds, such as a therapeutically effective amount of alpha-tocotrienol quinone, to an individual suffering from Ataxia-Telangiectasia (A-T), alleviates, arrests, or reverses the progression of, or prevents the occurrence of, telangiectasia. In another embodiment, administration of a therapeutically effective amount of alpha-tocotrienol quinone, alpha-tocotrienol hydroquinone, beta-tocotrienol quinone, beta-tocotrienol hydroquinone, gamma-tocotrienol quinone, gamma-tocotrienol hydroquinone, delta-tocotrienol quinone, delta-tocotrienol hydroquinone, or any combination of two or more of the foregoing compounds, such as a therapeutically effective amount of alpha-tocotrienol quinone, to an individual suffering from Ataxia-Telangiectasia (A-T), alleviates, arrests, or reverses the progression of, or prevents the occurrence of, sinopulmonary infections. In another embodiment, administration of a therapeutically effective amount of alpha-tocotrienol quinone, alpha-tocotrienol hydroquinone, beta-tocotrienol quinone, beta-tocotrienol hydroquinone, gamma-tocotrienol quinone, gamma-tocotrienol hydroquinone, delta-tocotrienol quinone, delta-tocotrienol hydroquinone, or any combination of two or more of the foregoing compounds, such as a therapeutically effective amount of alpha-tocotrienol quinone, to an individual suffering from Ataxia-Telangiectasia (A-T), alleviates, arrests, or reverses the progression of, or prevents, genomic instability. In another embodiment, administration of a therapeutically effective amount of alpha-tocotrienol quinone, alpha-tocotrienol hydroquinone, beta-tocotrienol quinone, beta-tocotrienol hydroquinone, gamma-tocotrienol quinone, gamma-tocotrienol hydroquinone, delta-tocotrienol quinone, delta-tocotrienol hydroquinone, or any combination of two or more of the foregoing compounds, such as a therapeutically effective amount of alpha-tocotrienol quinone, to an individual suffering from Ataxia-Telangiectasia (A-T), alleviates, arrests, or reverses the progression of, or prevents, the sensitivity to ionizing radiation such as X rays or gamma rays. In another embodiment, administration of a therapeutically effective amount of alpha-tocotrienol quinone, alpha-tocotrienol hydroquinone, beta-tocotrienol quinone, beta-tocotrienol hydroquinone, gamma-tocotrienol quinone, gamma-tocotrienol hydroquinone, delta-tocotrienol quinone, delta-tocotrienol hydroquinone, or any combination of two or more of the foregoing compounds, such as a therapeutically effective amount of alpha-tocotrienol quinone, to an individual suffering from Ataxia-Telangiectasia (A-T), alleviates, arrests, or reverses the progression of, or prevents the occurrence of, cancer or cancerous tumors. In another embodiment, administration of a therapeutically effective amount of alpha-tocotrienol quinone, alpha-tocotrienol hydroquinone, beta-tocotrienol quinone, beta-tocotrienol hydroquinone, gamma-tocotrienol quinone, gamma-tocotrienol hydroquinone, delta-tocotrienol quinone, delta-tocotrienol hydroquinone, or any combination of two or more of the foregoing compounds, such as a therapeutically effective amount of alpha-tocotrienol quinone, to an individual suffering from Ataxia-Telangiectasia (A-T), alleviates, arrests, or reverses the progression of, or prevents the occurrence of, leukemia. In another embodiment, administration of a therapeutically effective amount of alpha-tocotrienol quinone, alpha-tocotrienol hydroquinone, beta-tocotrienol quinone, beta-tocotrienol hydroquinone, gamma-tocotrienol quinone, gamma-tocotrienol hydroquinone, delta-tocotrienol quinone, delta-tocotrienol hydroquinone, or any combination of two or more of the foregoing compounds, such as a therapeutically effective amount of alpha-tocotrienol quinone, to an individual suffering from Ataxia-Telangiectasia (A-T), alleviates arrests or reverses the progression of, or prevents the occurrence of, lymphomas. In another embodiment, administration of a therapeutically effective amount of alpha-tocotrienol quinone, alpha-tocotrienol hydroquinone, beta-tocotrienol quinone, beta-tocotrienol hydroquinone, gamma-tocotrienol quinone, gamma-tocotrienol hydroquinone, delta-tocotrienol quinone, delta-tocotrienol hydroquinone, or any combination of two or more of the foregoing compounds, such as a therapeutically effective amount of alpha-tocotrienol quinone, to an individual suffering from Ataxia-Telangiectasia (A-T), alleviates, arrests, or reverses the progression of, or prevents the occurrence of, breast cancer.

In one embodiment, administration of a therapeutically effective amount of alpha-tocotrienol quinone, to an individual suffering from Ataxia-Telangiectasia (A-T), alleviates, arrests, or reverses the progression of, or prevents the occurrence of one or more symptoms selected from the group consisting of ataxia, telangiectasia, sinopulmonary infections, breast cancer, lymphomas and leukemia, genomic instability, and sensitivity to ionizing radiation such as X rays or gamma rays. In another embodiment, administration of a therapeutically effective amount of alpha-tocotrienol quinone, to an individual suffering from Ataxia-Telangiectasia (A-T), alleviates, arrests, or reverses the progression of, or prevents the occurrence of, ataxia. In another embodiment, administration of a therapeutically effective amount of alpha-tocotrienol quinone, to an individual suffering from Ataxia-Telangiectasia (A-T), alleviates, arrests, or reverses the progression of, or prevents the occurrence of, telangiectasia. In another embodiment, administration of a therapeutically effective amount of alpha-tocotrienol quinone, to an individual suffering from Ataxia-Telangiectasia (A-T), alleviates, arrests, or reverses the progression of, or prevents the occurrence of, sinopulmonary infections. In another embodiment, administration of a therapeutically effective amount of alpha-tocotrienol quinone, to an individual suffering from Ataxia-Telangiectasia (A-T), alleviates, arrests, or reverses the progression of, or prevents, genomic instability. In another embodiment, administration of a therapeutically effective amount of alpha-tocotrienol quinone, to an individual suffering from Ataxia-Telangiectasia (A-T), alleviates, arrests, or reverses the progression of, or prevents, sensitivity to ionizing radiation such as X rays or gamma rays. In another embodiment, administration of a therapeutically effective amount of alpha-tocotrienol quinone, to an individual suffering from Ataxia-Telangiectasia (A-T), alleviates, arrests, or reverses the progression of, or prevents the occurrence of, cancer or cancerous tumors. In another embodiment, administration of a therapeutically effective amount of alpha-tocotrienol quinone, to an individual suffering from Ataxia-Telangiectasia (A-T), alleviates, arrests, or reverses the progression of, or prevents the occurrence of, leukemia. In another embodiment, administration of a therapeutically effective amount of alpha-tocotrienol quinone, to an individual suffering from Ataxia-Telangiectasia (A-T), alleviates, arrests, or reverses the progression of, or prevents the occurrence of, lymphoma. In another embodiment, administration of a therapeutically effective amount of alpha-tocotrienol quinone, to an individual suffering from Ataxia-Telangiectasia (A-T) alleviates, arrests, or reverses the progression of, or prevents the occurrence of, breast cancer.

In one embodiment, administration of a therapeutically effective amount of one or more tocotrienol quinone, to an individual suffering from cancer or cancerous tumors associated with an Ataxia-Telangiectasia mutant deficiency, alleviates, arrests, or reverses the progression of, or prevents the occurrence of, said cancer or cancerous tumors. In one embodiment, administration of a therapeutically effective amount of alpha-tocotrienol quinone, to an individual suffering from cancer or cancerous tumors associated with an Ataxia-Telangiectasia mutant deficiency, alleviates, arrests, or reverses the progression of, or prevents the occurrence of, said cancer or cancerous tumors.

In one embodiment, administration of a therapeutically effective amount of one or more tocotrienol quinone, to an individual suffering from cancer or cancerous tumors associated with Ataxia-Telangiectasia mutant deficiency, alleviates, arrests, or reverses the progression of, or prevents the occurrence of, cancer or cancerous tumors displayed as, lymphomas and leukemia. In one embodiment, administration of a therapeutically effective amount of one or more tocotrienol quinone, to an individual suffering from cancer or cancerous tumors associated with Ataxia-Telangiectasia mutant deficiency alleviates, arrests, or reverses the progression of, or prevents the occurrence of, cancer or cancerous tumors displayed as breast cancer.

In one embodiment, administration of a therapeutically effective amount of alpha-tocotrienol quinone, to an individual suffering from cancer or cancerous tumors associated with Ataxia-Telangiectasia mutant deficiency, alleviates, arrests, or reverses the progression of, or prevents the occurrence of, cancer or cancerous tumors displayed as lymphomas and leukemia. In one embodiment, administration of a therapeutically effective amount of alpha-tocotrienol quinone, to an individual suffering from cancer or cancerous tumors associated with Ataxia-Telangiectasia mutant deficiency alleviates, arrests, or reverses the progression of, or prevents the occurrence of, cancer or cancerous tumors displayed as breast cancer.

In one embodiment, the compound used for treatment is administered to the patient in an amount such that the concentration of the compound in the plasma of the patient is between about 1 ng/ml and about 5,000 ng/ml. In one embodiment, the compound used for treatment is administered to the patient in an amount such that the concentration of the compound in the plasma of the patient is between about 1 ng/ml and about 2,000 ng/ml. In one embodiment, the compound used for treatment is administered to the patient in an amount such that the concentration of the compound in the plasma of the patient is between about 10 ng/ml and about 2,000 ng/ml. In one embodiment, the compound used for treatment is administered to the patient in an amount such that the concentration of the compound in the plasma of the patient is between about 10 ng/ml and about 1,000 ng/ml. In one embodiment, the compound used for treatment is administered to the patient in an amount such that the concentration of the compound in the plasma of the patient is between about 10 ng/ml and about 500 ng/ml. In one embodiment, the compound used for treatment is administered to the patient in an amount such that the concentration of the compound in the plasma of the patient is between about 10 ng/ml and about 250 ng/ml. In one embodiment, the compound used for treatment is administered to the patient in an amount such that the concentration of the compound in the plasma of the patient is between about 10 ng/ml and about 150 ng/ml. In one embodiment, the compound used for treatment is administered to the patient in an amount such that the concentration of the compound in the plasma of the patient is between about 10 ng/ml and about 100 ng/ml. In one embodiment, the compound used for treatment is administered to the patient in an amount such that the concentration of the compound in the plasma of the patient is about 50 ng/ml.

In one embodiment, the compound used for treatment is administered to the patient in an amount such that the concentration of the compound in the plasma of the patient is at or above about 1 ng/ml. In one embodiment, the compound used for treatment is administered to the patient in an amount such that the concentration of the compound in the plasma of the patient is at or above about 5 ng/ml. In one embodiment, the compound used for treatment is administered to the patient in an amount such that the concentration of the compound in the plasma of the patient is at or above about 10 ng/ml. In one embodiment, the compound used for treatment is administered to the patient in an amount such that the concentration of the compound in the plasma of the patient is at or above about 25 ng/ml. In one embodiment, the compound used for treatment is administered to the patient in an amount such that the concentration of the compound in the plasma of the patient is at or above about 50 ng/ml. In one embodiment, the compound used for treatment is administered to the patient in an amount such that the concentration of the compound in the plasma of the patient is at or above about 75 ng/ml. In one embodiment, the compound used for treatment is administered to the patient in an amount such that the concentration of the compound in the plasma of the patient is at or above about 100 ng/ml.

In one embodiment, the compound used for treatment is alpha-tocotrienol quinone, and the alpha-tocotrienol quinone is administered to the patient in an amount such that the concentration of alpha-tocotrienol quinone in the plasma of the patient is between about 1 ng/ml and about 5,000 ng/ml. In one embodiment, the compound used for treatment is alpha-tocotrienol quinone, and the alpha-tocotrienol quinone is administered to the patient in an amount such that the concentration of alpha-tocotrienol quinone in the plasma of the patient is between about 1 ng/ml and about 2,000 ng/ml. In one embodiment, the compound used for treatment is alpha-tocotrienol quinone, and the alpha-tocotrienol quinone is administered to the patient in an amount such that the concentration of alpha-tocotrienol quinone in the plasma of the patient is between about 10 ng/ml and about 2,000 ng/ml. In one embodiment, the compound used for treatment is alpha-tocotrienol quinone, and the alpha-tocotrienol quinone is administered to the patient in an amount such that the concentration of alpha-tocotrienol quinone in the plasma of the patient is between about 10 ng/ml and about 1,000 ng/ml. In one embodiment, the compound used for treatment is alpha-tocotrienol quinone, and the alpha-tocotrienol quinone is administered to the patient in an amount such that the concentration of alpha-tocotrienol quinone in the plasma of the patient is between about 10 ng/ml and about 500 ng/ml. In one embodiment, the compound used for treatment is alpha-tocotrienol quinone, and the alpha-tocotrienol quinone is administered to the patient in an amount such that the concentration of alpha-tocotrienol quinone in the plasma of the patient is between about 10 ng/ml and about 250 ng/ml. In one embodiment, the compound used for treatment is alpha-tocotrienol quinone, and the alpha-tocotrienol quinone is administered to the patient in an amount such that the concentration of alpha-tocotrienol quinone in the plasma of the patient is between about 10 ng/ml and about 150 ng/ml. In one embodiment, the compound used for treatment is alpha-tocotrienol quinone, and the alpha-tocotrienol quinone is administered to the patient in an amount such that the concentration of alpha-tocotrienol quinone in the plasma of the patient is between about 10 ng/ml and about 100 ng/ml. In one embodiment, the compound used for treatment is alpha-tocotrienol quinone, and the alpha-tocotrienol quinone is administered to the patient in an amount such that the concentration of alpha-tocotrienol quinone in the plasma of the patient is about 50 ng/ml.

In one embodiment, the compound used for treatment is alpha-tocotrienol quinone, and the alpha-tocotrienol quinone is administered to the patient in an amount such that the concentration of alpha-tocotrienol quinone in the plasma of the patient is at or above about 1 ng/ml. In one embodiment, the compound used for treatment is alpha-tocotrienol quinone, and the alpha-tocotrienol quinone is administered to the patient in an amount such that the concentration of alpha-tocotrienol quinone in the plasma of the patient is at or above about 5 ng/ml. In one embodiment, the compound used for treatment is alpha-tocotrienol quinone, and the alpha-tocotrienol quinone is administered to the patient in an amount such that the concentration of alpha-tocotrienol quinone in the plasma of the patient is at or above about 10 ng/ml. In one embodiment, the compound used for treatment is alpha-tocotrienol quinone, and the alpha-tocotrienol quinone is administered to the patient in an amount such that the concentration of alpha-tocotrienol quinone in the plasma of the patient is at or above about 25 ng/ml. In one embodiment, the compound used for treatment is alpha-tocotrienol quinone, and the alpha-tocotrienol quinone is administered to the patient in an amount such that the concentration of alpha-tocotrienol quinone in the plasma of the patient is at or above about 50 ng/ml. In one embodiment, the compound used for treatment is alpha-tocotrienol quinone, and the alpha-tocotrienol quinone is administered to the patient in an amount such that the concentration of alpha-tocotrienol quinone in the plasma of the patient is at or above about 75 ng/ml. In one embodiment, the compound used for treatment is alpha-tocotrienol quinone, and the alpha-tocotrienol quinone is administered to the patient in an amount such that the concentration of alpha-tocotrienol quinone in the plasma of the patient is at or above about 100 ng/ml.

In one embodiment, the compound for use in treating Ataxia-Telangiectasia (A-T) or Ataxia-telangiectasia like disorder (ATLD) is selected from the group consisting of alpha-tocotrienol quinone, beta-tocotrienol quinone, gamma-tocotrienol quinone, delta-tocotrienol quinone, alpha-tocotrienol hydroquinone, beta-tocotrienol hydroquinone, gamma-tocotrienol hydroquinone, and delta-tocotrienol hydroquinone, or any combination of two or more of the foregoing compounds, and is formulated in a pharmaceutical preparation suitable for oral administration, for example by spoon feeding, via feeding tube, via feeding syringe, or gastrostomy. In another embodiment, the compound for use in treating Ataxia-Telangiectasia (A-T) or Ataxia-telangiectasia like disorder (ATLD) is selected from the group consisting of alpha-tocotrienol quinone, beta-tocotrienol quinone, gamma-tocotrienol quinone, delta-tocotrienol quinone, alpha-tocotrienol hydroquinone, beta-tocotrienol hydroquinone, gamma-tocotrienol hydroquinone, and delta-tocotrienol hydroquinone, or any combination of two or more of the foregoing compounds, and is formulated in a pharmaceutical preparation comprising one or more vegetable-derived oils, such as sesame oil, and/or one or more animal-derived oils, and/or one or more fish-derived oils. In another embodiment, the compound for use in treating Ataxia-Telangiectasia (A-T) or Ataxia-telangiectasia like disorder (ATLD) is alpha-tocotrienol quinone, beta-tocotrienol quinone, gamma-tocotrienol quinone, delta-tocotrienol quinone, alpha-tocotrienol hydroquinone, beta-tocotrienol hydroquinone, gamma-tocotrienol hydroquinone, and delta-tocotrienol hydroquinone, or any combination of two or more of the foregoing compounds, and is formulated in a pharmaceutical preparation comprising one or more vegetable-derived oils, such as sesame oil, and/or one or more animal-derived oils, and/or one or more fish-derived oils, where the pharmaceutical preparation is suitable for oral administration by spoon feeding or via feeding tube, feeding syringe, or gastrostomy.

For all of the compounds and methods described herein which use a tocotrienol quinone, the quinone form can also be used in its reduced (hydroquinone, 1,4-benzenediol) form when desired. Likewise, the hydroquinone form can also be used in its oxidized (quinone) form when desired.

For all of the compounds and methods described herein, the invention also encompasses the use in treatment of the compounds and methods disclosed. The invention also encompasses the use of the compounds described herein for preparation of a medicament for use in treating Ataxia-Telangiectasia (A-T). The invention also encompasses the use of the compounds described herein for preparation of a medicament for use in treating Ataxia-telangiectasia like disorder (ATLD).

The present invention comprises multiple aspects, features and embodiments, where such multiple aspects, features and embodiments can be combined and permuted in any desired manner. These and other aspects, features and embodiments of the present invention will become evident upon reference to the remainder of this application, including the following detailed description. In addition, various references are set forth herein that describe in more detail certain compositions, and/or methods; all such references are incorporated herein by reference in their entirety.

### DETAILED DESCRIPTION OF THE INVENTION

The present invention relates to a method of treating Ataxia-Telangiectasia (A-T) or Ataxia-telangiectasia like disorder (ATLD), with specific compounds.

In one aspect, tocotrienol quinones are contemplated for use in treatment, including alpha-tocotrienol quinone, beta-tocotrienol quinone, gamma-tocotrienol quinone, delta-tocotrienol quinone and/or any combination of two or more of the foregoing compounds. In another aspect, alpha-tocotrienol quinone is contemplated for use in treatment. Structures of tocotrienol quinones are given in Table 1 below. The tocotrienol quinones with the naturally occurring tocotrienol configuration are used in one embodiment of the invention, but other stereoisomers and/or mixtures of stereoisomers in any ratio, such as racemic mixtures, can also be used in the invention.

Tocotrienol quinones can be used in their oxidized form, as shown in Table 1, or can be used in their reduced hydroquinone form, as shown in Table 2. The quinone (cyclohexadienedione) form and hydroquinone (benzenediol) form are readily interconverted with appropriate reagents. The quinone can be treated in a biphasic mixture of an ethereal solvent with a basic aqueous solution of Na₂S₂O₄ (Vogel, A. I. et al. Vogel's Textbook of Practical Organic Chemistry, 5^{th} Edition, Prentice Hall: New York, 1996; Section 9.6.14 Quinones, “Reduction to the Hydroquinone”). Standard workup in the absence of oxygen yields the desired hydroquinone. The hydroquinone form can be oxidized to the quinone form with oxidizing agents such as ceric ammonium nitrate (CAN) or ferric chloride. The quinone and hydroquinone forms are also readily interconverted electrochemically, as is well known in the art. See, e.g., Section 33.4 of Streitweiser & Heathcock, Introduction to Organic Chemistry, New York: Macmillan, 1976.

TABLE 1
Tocotrienol quinones

| 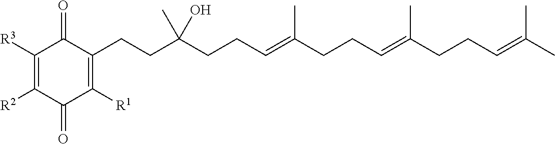 | | R¹ | R² | R³ |
|---|---|---|---|---|
| Alpha- tocotrienol quinone | 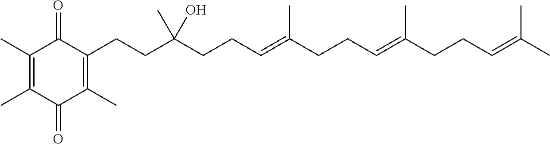 [SMILES: CC(C)=CCC/C(C)=C/CC/C(C)=C/CCC(C)(O)CCC1=C(C)C(=O)C(C)=C(C)C1=O] | methyl | methyl | methyl |
| Beta-tocotrienol quinone | 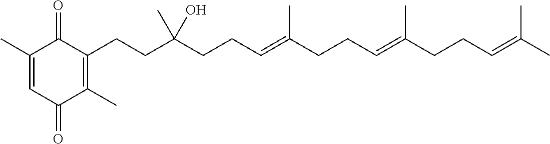 [SMILES: CC(C)=CCC/C(C)=C/CC/C(C)=C/CCC(C)(O)CCC1=C(C)C(=O)C=C(C)C1=O] | methyl | H | methyl |
| Gamma- tocotrienol quinone | 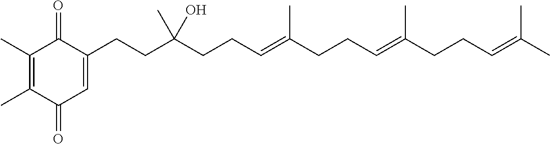 [SMILES: CC(C)=CCC/C(C)=C/CC/C(C)=C/CCC(C)(O)CCC1=CC(=O)C(C)=C(C)C1=O] | H | methyl | methyl |
| Delta-tocotrienol quinone | 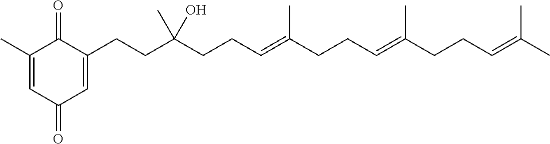 | H | H | methyl |

TABLE 2
Tocotrienol hydroquinones

| 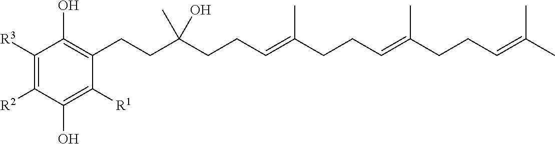 | | R¹ | R² | R³ |
|---|---|---|---|---|
| Alpha-tocotrienol hydroquinone | 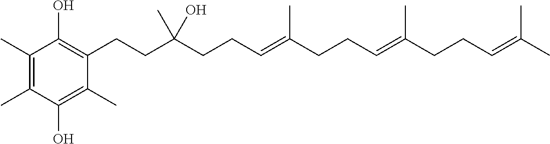 [SMILES: CC(C)=CCC/C(C)=C/CC/C(C)=C/CCC(C)(O)CCc1c(C)c(O)c(C)c(C)c1O] | methyl | methyl | methyl |
| Beta-tocotrienol hydroquinone | 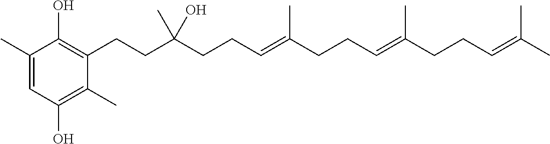 [SMILES: CC(C)=CCC/C(C)=C/CC/C(C)=C/CCC(C)(O)CCc1c(C)c(O)cc(C)c1O] | methyl | H | methyl |
| Gamma- tocotrienol hydroquinone | 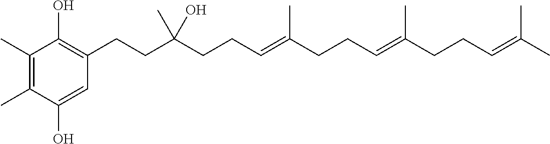 [SMILES: CC(C)=CCC/C(C)=C/CC/C(C)=C/CCC(C)(O)CCc1cc(O)c(C)c(C)c1O] | H | methyl | methyl |
| Delta-tocotrienol hydroquinone | 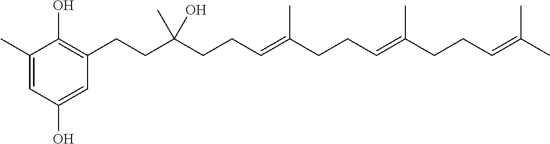 | H | H | methyl |

By “individual,” “subject,” or “patient,” is meant a mammal, preferably a human.

“Treating” a disease with the compounds and methods discussed herein is defined as administering one or more of the compounds discussed herein, with or without additional therapeutic agents, in order to reduce or eliminate either the disease or one or more symptoms of the disease, or to retard the progression of the disease or of one or more symptoms of the disease, or to reduce the severity of the disease or of one or more symptoms of the disease. “Suppression” of a disease with the compounds and methods discussed herein is defined as administering one or more of the compounds discussed herein, with or without additional therapeutic agents, in order to suppress the clinical manifestation of the disease, or to suppress the manifestation of adverse symptoms of the disease. The distinction between treatment and suppression is that treatment occurs after adverse symptoms of the disease are manifest in a subject, while suppression occurs before adverse symptoms of the disease are manifest in a subject. Suppression may be partial, substantially total, or total.

Because Ataxia-Telangiectasia (A-T) and Ataxia-telangiectasia like disorder (ATLD) are due to genetic mutations, genetic screening can be used to identify patients at risk of the disease. Ataxia-Telangiectasia (A-T) or Ataxia-telangiectasia like disorder (ATLD) can arise from mutations in the ATM or hMre11 gene. The compounds disclosed herein can be administered to, and the methods of the invention disclosed herein can be used to treat, asymptomatic patients with mutations in the ATM or hMre11 gene, who are at risk of developing the clinical symptoms of the disease, in order to suppress the occurrence of any adverse symptoms or lessen the severity of symptoms that may occur. The compounds disclosed herein can be administered to, and the methods of the invention disclosed herein can be used to treat, symptomatic patients with mutations in the ATM or hMre11 gene, in order to treat the disease.

“Therapeutic use” of the compounds discussed herein is defined as using one or more of the compounds discussed herein to treat or suppress a disease, as defined above. A “therapeutically effective amount” of a compound is an amount of the compound, which, when administered to a subject, is sufficient to reduce or eliminate either a disease or one or more symptoms of a disease, or to retard the progression of a disease or of one or more symptoms of a disease, or to reduce the severity of a disease or of one or more symptoms of a disease, or to suppress the clinical manifestation of a disease, or to suppress the manifestation of adverse symptoms of a disease. A therapeutically effective amount can be given in one or more administrations.

While the compounds described herein can occur and can be used as the neutral (non-salt) compound, the description is intended to embrace all salts of the compounds described herein, as well as methods of using such salts of the compounds. In one embodiment, the salts of the compounds comprise pharmaceutically acceptable salts. Pharmaceutically acceptable salts are those salts which can be administered as drugs or pharmaceuticals to humans and/or animals and which, upon administration, retain at least some of the biological activity of the free compound (neutral compound or non-salt compound). The desired salt of a basic compound may be prepared by methods known to those of skill in the art by treating the compound with an acid. Examples of inorganic acids include, but are not limited to, hydrochloric acid, hydrobromic acid, sulfuric acid, nitric acid, and phosphoric acid. Examples of organic acids include, but are not limited to, formic acid, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, sulfonic acids, and salicylic acid. Salts of basic compounds with amino acids, such as aspartate salts and glutamate salts, can also be prepared. The desired salt of an acidic compound can be prepared by methods known to those of skill in the art by treating the compound with a base. Examples of inorganic salts of acid compounds include, but are not limited to, alkali metal and alkaline earth salts, such as sodium salts, potassium salts, magnesium salts, and calcium salts; ammonium salts; and aluminum salts. Examples of organic salts of acid compounds include, but are not limited to, procaine, dibenzylamine, N-ethylpiperidine, N,N-dibenzylethylenediamine, and triethylamine salts. Salts of acidic compounds with amino acids, such as lysine salts, can also be prepared.

The description of compounds herein also includes all stereoisomers of the compounds, including diastereomers and enantiomers, and mixtures of stereoisomers in any ratio, including, but not limited to, racemic mixtures. Unless stereochemistry is explicitly indicated in a structure, the structure is intended to embrace all possible stereoisomers of the compound depicted. If stereochemistry is explicitly indicated for one portion or portions of a molecule, but not for another portion or portions of a molecule, the structure is intended to embrace all possible stereoisomers for the portion or portions where stereochemistry is not explicitly indicated.

The compounds can be administered in prodrug form. Prodrugs are derivatives of the compounds, which are themselves relatively inactive but which convert into the active compound when introduced into the subject in which they are used by a chemical or biological process in vivo, such as an enzymatic conversion. Suitable prodrug formulations include, but are not limited to, peptide conjugates of the compounds disclosed herein and esters of compounds disclosed herein. Further discussion of suitable prodrugs is provided in H. Bundgaard, Design of Prodrugs, New York: Elsevier, 1985; in R. Silverman, The Organic Chemistry of Drug Design and Drug Action, Boston: Elsevier, 2004; in R. L. Juliano (ed.), Biological Approaches to the Controlled Delivery of Drugs (Annals of the New York Academy of Sciences, v. 507), New York: New York Academy of Sciences, 1987; and in E. B. Roche (ed.), Design of Biopharmaceutical Properties Through Prodrugs and Analogs (Symposium sponsored by Medicinal Chemistry Section, APhA Academy of Pharmaceutical Sciences, November 1976 national meeting, Orlando, Fla.), Washington: The Academy, 1977.

The description of compounds herein also includes all isotopologues (molecular entities that differs only in isotopic composition) of the compounds described herein, and mixtures of isotopologues in any ratio.

### Laboratory Findings and Measurements

Laboratory findings include: (1) elevated serum alpha-fetoprotein; (2) immunological deficiencies such as low T cell levels, poor in vitro responses to mitogens, low serum levels of IgA, IgE and IgG2, and poor in vivo responses to pneumococcal polysaccharides; (3) characteristic chromosomal aberrations such as t(7;14) translocations and telomeric fusions, and an increased rate of telomeric shortening; (4) in vitro radiosensitivity, expressed as reduced colony forming ability following exposure to ionizing radiation or radiomimetic chemicals; (5) profound defects in the activation of cell cycle checkpoints, such as radioresistant DNA synthesis; (6) absent or decreased intracellular ATM levels by Western blotting; (7) deficient phosphorylation of many substrates, such as p53, nibrin/Nbs1,Mdm2, Smc1, Mre11 and ATM itself (autophosphorylation at serine 1981); and (8) mutations in the ATM gene. (See Chun, H H et al., DNA Repair (2004) 3:1187-1196.)

The colony survival assay (CSA) is the only measure of radiosensitivity that has been validated for clinical use (Y. K. Cancer Res. (1994) 54:2544-2547). CSA measures the survival fraction of lymphoblastoid cell lines established from patient samples following exposure to 1 Gray gamma radiation. Survival fractions for classical A-T usually score below 21% signifying radiosensitivity. A normal response to irradiation is >36%. A-T cells also exhibit radioresistant DNA synthesis due to abnormal S phase, propagating/fixing DNA damage into the genome. A-T cells also arrest abnormally at G2/M of the cell cycle. (Chun, H H et al., DNA Repair (2004) 3:1187-1196.)

### Ataxia-Telangiectasia (A-T) and Ataxia-Telangiectasia Like Disorder (ATLD): Symptoms Amenable to Treatment

Ataxia-telangiectasia (A-T) gives rise to several devastating symptoms, including truncal ataxia (difficulty with control of body posture and body movement); peripheral ataxia (abnormal coordination of limbs); cerebellar ataxia; chorea (small jerks of the hands and feet which look like fidgeting); atheosis (slower twisting movements of the upper body); dystonia (adoption of stiff and twisted postures); myoclonic jerks (occasional uncontrolled jerks); swallowing dysfunction; tremors (shaking episodes of a limb which are like shivering); dysarthria (slurring of speech); vertical and horizontal sacchadic apraxia (restricted eye movements); telangiectasias (prominent blood vessels in the whites of the eyes or in the facial skin); immunodeficiency symptoms such as sinopulmonary infections (repeated colds and runny noses); cancer or cancerous tumors including lymphomas, leukemia, and breast cancer; increased sensitivity to ionizing radiation (X rays and gamma rays); thymic hypoplasia; hypogonadism; genomic instability; and premature aging symptoms such as diabetes mellitus and progeria (hair graying, hair loss, wrinkling of the skin, bone deterioration, premature cataracts, and need for reading glasses in children).

Symptoms of Ataxia-telangiectasia like disorder (ATLD) are similar to those of Ataxia-telangiectasia (A-T), although they may not be as severe, and do have the absence of telangiectasias, normal immunoglobin levels, a later onset of the condition, and a slower progression of the disease.

In one embodiment, the methods of the invention can alleviate one or more symptoms of Ataxia-Telangiectasia (A-T) or Ataxia-telangiectasia like disorder (ATLD), including truncal ataxia (loss of control of body posture and body movement); peripheral ataxia (abnormal coordination of limbs); cerebellar ataxia; chorea (small jerks of the hands and feet which look like fidgeting); atheosis (slower twisting movements of the upper body); dystonia (adoption of stiff and twisted postures); myoclonic jerks (occasional uncontrolled jerks); swallowing dysfunction; tremors (shaking episodes of a limb which are like shivering); dysarthria (slurring of speech); vertical and horizontal sacchadic apraxia (restricted eye movements); telangiectasias (prominent blood vessels in the whites of the eyes or in the facial skin); immunodeficiency symptoms such as sinopulmonary infections (repeated colds and runny noses); cancer or cancerous tumors including lymphomas, leukemia, and breast cancer; increased sensitivity to ionizing radiation (X rays and gamma rays); thymic hypoplasia; hypogonadism; genomic instability; and premature aging symptoms such as diabetes mellitus and progeria (hair graying, hair loss, wrinkling of the skin, bone deterioration, premature cataracts, and need for reading glasses in children).

In one embodiment, the methods of the invention can alleviate one or more symptoms of Ataxia-Telangiectasia (A-T) or Ataxia-telangiectasia like disorder (ATLD), including ataxia, telangiectasia, sinopulmonary infections, lymphomas, leukemia, breast cancer, genomic instability, and sensitivity to ionizing radiation such as X rays or gamma rays.

In another embodiment, treatment according to the invention can produce in a patient an adequate reduction or alleviation of one or more of the observable characteristics of Ataxia-Telangiectasia (A-T) by an amount that is discernible to a human observer, such as a parent, physician or caretaker, without the use of special devices such as imaging technology, microscopes or chemical analytical devices. For example, treatment according to the invention can produce an observable reduction of ataxia and difficulty in walking, wherein a patient that was bed-bound and lethargic prior to treatment is able, after treatment, to walk with assistance; or can enable balancing, including balancing on one foot; riding a tricycle; walking up steps; sitting without assistance; independently standing and supporting himself or herself by holding on to a table or a fixed object for at least one minute; turning and scooting or sliding while sitting; moving his or her extremities purposefully, as in giving a “high-five” gesture; and performing fine motor tasks such as grasping small objects. Treatment according to the invention can produce an observable reduction of speech problems, such as speaking in complete sentences, improved enunciation, counting aloud, having increased voice and word association. In another example, treatment according to the invention can produce an observable reduction of telangiectasia.

Standard motor function tests can be used to assess many of these symptoms, including tests used by physical therapists, occupational therapists, and rehabilitation medicine specialists to assess patient function. As many patients presenting with Ataxia-Telangiectasia (A-T) or Ataxia-telangiectasia like disorder (ATLD) are young, age-appropriate tests are used.

There are several known assessment products for pediatricians to evaluate children. For physical abilities, the Pediatric Evaluation of Disability Inventory (PEDI) can be used (see Haley, S. M., Coster, W. J., Ludlow, L. H., Haltiwanger, J. T., & Andrellos, P. J. (1992). Pediatric Evaluation of Disability Inventory: Development, Standardization, and Administration Manual, Version 1.0. Boston, Mass.: Trustees of Boston University, Health and Disability Research Institute); PEDI enables evaluation of functional disabilities using standardized score forms. The PEDI can be used to assess key functional capabilities and performance in children ages six months to seven years, and to evaluate older children whose functional abilities are lower than those of seven-year-olds without disabilities. PEDI can be used to identify functional deficits and monitor treatment progress.

For neuro-psychiatric evaluation, the NEPSY-II assessment (Korkman, Marit; Kirk, Ursula; & Kemp, Sally. (2007) NEPSY-II-Second Edition, San Antonio, Tex.: Pearson) can be used to gauge neuropsychological development. Testing in children 3-4 years of age can assess six functional domains: attention and executive functions; language and communication; sensorimotor functions; visuospatial functions; learning and memory; and social perception.

In one embodiment, the method of the invention can alleviate one or more symptoms of A-T or ATLD, such as in a patient suffering from A-T or ATLD by enhancing the ATM response in cells, which is involved in DNA damage sensing and repair; modulating cellular response to oxidation, which affects the other signaling pathways; and altering usage of metabolic energy pathways. In some embodiments the methods of the invention can promote cell death in cancer cells in a subject suffering from cancer or cancerous tumors associated with A-T or ATLD. In some embodiments the methods of the invention can promote cell death in cancer cells from a patient suffering from A-T or ATLD, wherein the cancer cells are lymphoma or leukemia cells. In some embodiments the methods of the invention can promote cell death in cancer cells from a patient suffering from A-T or ATLD, wherein the cancer cells are breast cancer cells. In other embodiments the methods of the invention can inhibit the progression of metastases of cancer cells in a subject suffering from cancer associated with A-T or ATLD. In other embodiments the methods of the invention can inhibit the progression of metastases of cancer cells from a patient suffering from A-T or ATLD, wherein the cancer cells are lymphoma cells. In other embodiments the methods of the invention can inhibit the progression of metastases of cancer cells from a patient suffering from A-T or ATLD, wherein the cancer cells are leukemia cells. In other embodiments the methods of the invention can inhibit the progression of metastases of cancer cells from a patient suffering from A-T or ATLD, wherein the cancer cells are breast cancer cells.

In another embodiment, the method of the invention can alleviate cancer or cancerous tumors resulting from an ATM deficiency, including cancer or cancerous tumors which compromise one or more cancer cells which, compared to normal cells, have a reduced ability or an inability to repair DNA damage through the ATM-dependent DNA damage pathway. In some embodiments, the number, volume, or weight of cancerous cells resulting from ATM-dependent DNA damage is reduced by about 10% or more, about 20% or more, about 30% or more, about 40% or more, about 50% or more, about 60% or more, about 70% or more, about 80% or more, or about 90% or more.

The efficacy of tocotrienol quinones in their ability to alleviate, arrest the progression of, or reverse the occurrence of tumors in humans with A-T or ATLD can be assessed in tumor prone ATM-deficient mice. Two examples of mutant mice include but are not limited to the ATM^{tm1Mfl}/ATM^{tm1Mfl} mouse, also named ATM-DeltaSRI or DeltaSRI (MGI:2181756, involving strain origin 129T2/SvEms*C57BL/6J) (Spring K. et al “ATM knock-in mice harboring an in-frame deletion corresponding to the human ATM 7636del9 common mutation exhibit a variant phenotype”, Cancer Res. (2001) June 1; 61(11):4561-8) or the ATM^{tm1Awb}/ATM^{tm1Awb} mouse (JAX:002753, involving the strain origin 129S2/SvEvTac) (Barlow C et al, “ATM-deficient mice: a paradigm of ataxia telangiectasia” Cell (2003) 86(1):159-71).

Other examples of murine models for ataxia-telangiectasia include, but are not limited to, models of ataxia-telangiectasia as described in Barlow et al., 1999, Proc Natl Acad Sci USA 96(17):9915-9 and Inoue et al., 1986, Cancer Res 46(8):3979-82; or a mouse model generated for ataxia-telangiectasia using gene targeting to generate mice that do not express the ATM protein, as described in Elson et al., 1996, Proc. Nat. Acad. Sci. 93: 13084-13089.

### Mutations Causing Ataxia-Telangiectasia

Several mutations in genes involved in energy metabolism are implicated in Ataxia-Telangiectasia (A-T) or Ataxia-telangiectasia like disorder (ATLD). The genes involved in Ataxia-Telangiectasia (A-T) mutations have been identified to occur in chromosome 11q22-23. The genes involved in Ataxia-telangiectasia like disorder (ATLD) mutations have been identified to occur in chromosome 11q21. Null mutations in the ATM gene cause complete loss of function of the protein and are inherited in a recessive manner. Missense mutations produce stable, full size protein with reduced function such as substitutions, short in-frame insertions and deletions, and act by interfering with the normal copy of the protein. Missense mutations are most commonly found in carriers. Individuals with two missense mutations have a milder form of Ataxia-Telangiectasia.

Individuals with mutations in these genes who do not presently manifest symptoms of Ataxia-Telangiectasia (A-T) or Ataxia-telangiectasia like disorder (ATLD) can be treated with the methods of the invention in order to suppress symptoms of Ataxia-Telangiectasia (A-T) or Ataxia-telangiectasia like disorder (ATLD), or to lessen the severity of symptoms of Ataxia-Telangiectasia (A-T) or Ataxia-telangiectasia like disorder (ATLD) once they develop. Accordingly, in one aspect, the invention comprises methods of administering specific compounds, such as tocotrienol quinones, to individuals who have one or more of the mutations listed herein. In another aspect, the invention comprises methods of administering alpha-tocotrienol quinone to individuals who have one or more of the mutations listed herein.

### Dosages

The compounds used in the methods of the invention can be administered in various amounts. Examples of daily dosages which can be used are an effective amount within the dosage range of about 0.1 mg/kg to about 300 mg/kg body weight, or within about 0.1 mg/kg to about 100 mg/kg body weight, or within about 0.1 mg/kg to about 80 mg/kg body weight, or within about 0.1 mg/kg to about 50 mg/kg body weight, or within about 0.1 mg/kg to about 30 mg/kg body weight, or within about 0.1 mg/kg to about 10 mg/kg body weight, or within about 1.0 mg/kg to about 80 mg/kg body weight, or within about 1.0 mg/kg to about 50 mg/kg body weight, or within about 1.0 mg/kg to about 30 mg/kg body weight, or within about 1.0 mg/kg to about 10 mg/kg body weight, or within about 10 mg/kg to about 80 mg/kg body weight, or within about 50 mg/kg to about 150 mg/kg body weight, or within about 100 mg/kg to about 200 mg/kg body weight, or within about 150 mg/kg to about 250 mg/kg body weight, or within about 200 mg/kg to about 300 mg/kg body weight, or within about 250 mg/kg to about 300 mg/kg body weight, or about 0.1, about 5, about 10, about 15, about 20, about 25, about 30, about 40, about 50, about 60, about 70, about 75, about 80, about 90, about 100, about 125, about 150, about 175, about 200, about 225, about 250, about 275, about 300, about 325, about 350, about 375, about 400, about 425, about 450, about 500, about 550, about 600, about 650, about 700, about 750, about 800, about 850, about 900, about 950, or about 1000 mg total. The compound(s) may be administered in a single daily dose, or the total daily dosage may be administered in divided dosage of two, three or four times daily. These dosages can be administered long term, for example, over months, years, or even over the entire lifetime of the patient.

The particular dosage appropriate for a specific patient is determined by dose titration. For example, animal studies of alpha-tocotrienol quinone administration have shown that in rats, at 10 mg/kg, bioavailability is high (˜90%), Cₘₐₓ=931 ng/mL, Tₘₐₓ=3.5 h and t_{1/2}=3.5 h. There is less than dose-proportionality since for an increase in doses of 2.4:6:10:20 there is only an increase in AUCs of 1.5:2.8:4.0:6.7. This lack of dose-proportionality may be due to decreased absorption since there is no change in t_{1/2} over dose range. Alpha-tocotrienol quinone tested in rats was safe when given acutely up to 2000 mg/kg. In fasted dogs, at 10 mg/kg, bioavailability is low (˜16%), Cₘₐₓ=442 ng/mL, Tₘₐₓ=2.8 h and t1/2=7.6 h.

The single dose and repeat dose plasma profiles for alpha tocotrienol quinone were simulated using a dose adjusted to achieve a Cₘₐₓ<10 μM and a Cₘᵢₙ>0.5 μM. Assuming a daily dose and linear kinetics, for a 70 kg adult the total dose would need to be 379 mg (5.41 mg/kg) to achieve a C₂₄ₕ of 220.5 ng/ml (0.5 μM). The dose is adjusted as appropriate, as many patients with Ataxia-Telangiectasia (A-T) or Ataxia-telangiectasia like disorder (ATLD) are children weighing much less than 70 kg.

The starting dose can be estimated based on the United States Food and Drug Administration guidelines titled “Estimating the Maximum Safe Starting Dose in Initial Clinical Trials for Therapeutics in Adult Healthy Volunteers” (July 2005) as well as the International Conference on Harmonisation of Technical Requirements for Registration of Pharmaceuticals for Human Use (ICH) guidelines titled “Guidance on Non-clinical Safety Studies for the Conduct of Human Clinical Trials and Marketing Authorization for Pharmaceuticals” (July 2008). Per ICH guidelines, predicted exposures from the starting dose should not exceed 1/50^{th} the NOAEL (No-Adverse-Observed-Effect-Level) in the more sensitive species on a mg/m² basis. Following a single oral dose of alpha-tocotrienol quinone, the NOAEL was established to be 500 mg/kg for the female rat, i.e. 3,000 mg/m2. This dosage would be equivalent to 81 mg/kg in an adult human. 1/50th of 81 mg/kg is 1.6 mg/kg, i.e. 110 mg for a 70 kg adult, or 16 mg for a 10 kg child. This dose can be administered once, twice, or three times daily.

### Co-Administered Agents

While the compounds described herein can be administered as the sole active pharmaceutical agent, they can also be used in combination with one or more other agents used in the treatment or suppression of Ataxia-Telangiectasia (A-T) or Ataxia-telangiectasia like disorder (ATLD). Representative agents useful in combination with the compounds described herein for the treatment or suppression of Ataxia-Telangiectasia (A-T) or Ataxia-telangiectasia like disorder (ATLD) include, but are not limited to, Coenzyme Q, including Coenzyme Q10; reduced Coenzyme Q including reduced Coenzyme Q10; idebenone; MitoQ; acetylcarnitine (such as acetyl-L-carnitine or acetyl-DL-carnitine); palmitoylcarnitine (such as palmitoyl-L-carnitine or palmitoyl-DL-carnitine); carnitine (such as L-carnitine or DL-carnitine); quercetine; mangosteen; acai; uridine; N-acetyl cysteine (NAC); polyphenols, such as resveratrol; Vitamin A; Vitamin C; lutein; beta-carotene; lycopene; glutathione; fatty acids, including omega-3 fatty acids such as a-linolenic acid (ALA), eicosapentaenoic acid (EPA), and docosahexaenoic acid (DHA); lipoic acid; and lipoic acid derivatives; Vitamin B complex; Vitamin B1 (thiamine); Vitamin B2 (riboflavin); Vitamin B3 (niacin, nicotinamide, or niacinamide); Vitamin B5 (pantothenic acid); Vitamin B6 (pyridoxine or pyridoxamine); Vitamin B7 (biotin); Vitamin B9 (folic acid, also known as Vitamin B11 or Vitamin M); Vitamin B12 (cobalamins, such as cyanocobalamin); inositol; 4-aminobenzoic acid; folinic acid; Vitamin E; other vitamins; and antioxidant compounds.

The co-administered agents can be administered simultaneously with, prior to, or after, administration of the primary compound intended to treat Ataxia-Telangiectasia (A-T) or Ataxia-telangiectasia like disorder (ATLD).

### Formulations and Routes of Administration

The compounds used in the methods of the invention may be administered in any suitable form that will provide sufficient plasma and/or central nervous system levels of the compounds. The compounds can be administered enterally, orally, parenterally, sublingually, by inhalation (e.g. as mists or sprays), rectally, or topically in unit dosage formulations containing conventional nontoxic pharmaceutically acceptable carriers, excipients, adjuvants, and vehicles as desired. For example, suitable modes of administration include oral, subcutaneous, transdermal, transmucosal, iontophoretic, intravenous, intraarterial, intramuscular, intraperitoneal, intranasal (e.g. via nasal mucosa), subdural, rectal, gastrointestinal, and the like, and directly to a specific or affected organ or tissue. For delivery to the central nervous system, spinal and epidural administration, or administration to cerebral ventricles, can be used. Topical administration may also involve the use of transdermal administration such as transdermal patches or iontophoresis devices. The term parenteral as used herein includes subcutaneous injections, intravenous injection, intraarterial injection, intramuscular injection, intrasternal injection, or infusion techniques. The compounds are mixed with pharmaceutically acceptable carriers, excipients, adjuvants, and vehicles appropriate for the desired route of administration.

In certain embodiments of the invention, especially those embodiments where a formulation is used for injection or other parenteral administration, including the routes listed herein, but also including embodiments used for oral, gastric, gastrointestinal, or enteric administration, the formulations and preparations used in the methods of the invention are sterile. Sterile pharmaceutical formulations are compounded or manufactured according to pharmaceutical-grade sterilization standards (United States Pharmacopeia Chapters 797, 1072, and 1211; California Business & Professions Code 4127.7; 16 California Code of Regulations 1751, 21 Code of Federal Regulations 211) known to those of skill in the art.

Oral administration is advantageous due to its ease of implementation and patient (or caretaker) compliance. It is advised that the compounds be administered with a fatty food of the patient's choice such as yogurt or ice cream to improve drug absorbance. However, patients with Ataxia-Telangiectasia (A-T) or Ataxia-telangiectasia like disorder (ATLD) often have difficulty in swallowing. Introduction of medicine via feeding tube, feeding syringe, or gastrostomy can be employed in order to accomplish enteric administration. The active compound (and, if present, other co-administered agents) can be enterally administered in sesame oil, or any other pharmaceutically acceptable carrier suitable for formulation for administration via feeding tube, feeding syringe, or gastrostomy.

The term “nutraceutical” has been used to refer to any substance that is a food or a part of a food and provides medical or health benefits, including the prevention and treatment of disease. Hence, compositions falling under the label “nutraceutical” may range from isolated nutrients, dietary supplements and specific diets to genetically engineered designer foods, herbal products, and processed foods such as cereals, soups and beverages. In a more technical sense, the term has been used to refer to a product isolated or purified from foods, and generally sold in medicinal forms not usually associated with food and demonstrated to have a physiological benefit or provide protection against chronic disease. Accordingly, the compounds described for use herein can also be administered as nutraceutical or nutritional formulations, with additives such as nutraceutically or nutritionally acceptable excipients, nutraceutically or nutritionally acceptable carriers, and nutraceutically or nutritionally acceptable vehicles. Such formulations are sometimes called medical foods. Suitable nutraceutically acceptable excipients may include liquid solutions such as a solution comprising one or more vegetable-derived oils, such as sesame oil, and/or one or more animal-derived oils, and/or one or more fish-derived oils.

The compounds described for use herein can be administered in solid form, in liquid form, in aerosol form, or in the form of tablets, pills, powder mixtures, capsules, granules, injectables, creams, solutions, suppositories, enemas, colonic irrigations, emulsions, dispersions, food premixes, and in other suitable forms. The compounds can also be administered in liposome formulations. The compounds can also be administered as prodrugs, where the prodrug undergoes transformation in the treated subject to a form which is therapeutically effective. Additional methods of administration are known in the art.

Injectable preparations, for example, sterile injectable aqueous or oleaginous suspensions, may be formulated according to methods known in the art using suitable dispersing or wetting agents and suspending agents. The sterile injectable preparation may also be a sterile injectable solution or suspension in a nontoxic parenterally acceptable diluent or solvent, for example, as a solution in propylene glycol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono or di-glycerides. In addition, fatty acids such as oleic acid find use in the preparation of injectables.

Solid dosage forms for oral administration may include capsules, tablets, pills, powders, and granules. In such solid dosage forms, the active compound may be admixed with at least one inert diluent such as sucrose, lactose, or starch. Such dosage forms may also comprise additional substances other than inert diluents, e.g., lubricating agents such as magnesium stearate. In the case of capsules, tablets, and pills, the dosage forms may also comprise buffering agents. Tablets and pills can additionally be prepared with enteric coatings.

Liquid dosage forms for oral administration may include pharmaceutically acceptable emulsions, solutions, suspensions, syrups, and elixirs containing inert diluents commonly used in the art, such as water. Such compositions may also comprise adjuvants, such as wetting agents, emulsifying and suspending agents, cyclodextrins, and sweetening, flavoring, and perfuming agents. Alternatively, the compound may also be administered in neat form if suitable.

The compounds for use in the present invention can also be administered in the form of liposomes. As is known in the art, liposomes are generally derived from phospholipids or other lipid substances. Liposomes are formed by mono or multilamellar hydrated liquid crystals that are dispersed in an aqueous medium. Any non-toxic, physiologically acceptable and metabolizable lipid capable of forming liposomes can be used. The present compositions in liposome form can contain, in addition to a compound for use in the present invention, stabilizers, preservatives, excipients, and the like. The preferred lipids are the phospholipids and phosphatidyl cholines (lecithins), both natural and synthetic. Methods to form liposomes are known in the art. See, for example, Prescott, Ed., Methods in Cell Biology, Volume XIV, Academic Press, New York, N.W., p. 33 et seq (1976).

The amount of active ingredient that may be combined with the carrier materials to produce a single dosage form can vary depending upon the patient to which the active ingredient is administered and the particular mode of administration. It will be understood, however, that the specific dose level for any particular patient will depend upon a variety of factors including the activity of the specific compound employed; the age, body weight, body area, body mass index (BMI), general health, sex, and diet of the patient; the time of administration and route of administration used; the rate of excretion; drug combination, if any, used; and the progression, and severity of the disease in the patient undergoing therapy. The pharmaceutical unit dosage chosen is usually fabricated and administered to provide a defined final concentration of drug in the blood, cerebrospinal fluid, brain tissues, spinal cord tissues, other tissues, other organs, or other targeted region of the body.

Compounds for use in the present invention may be administered in a single daily dose, or the total daily dosage may be administered in divided dosage of two, three or four times daily.

While the compounds for use in the present invention can be administered as the sole active pharmaceutical agent, they can also be used in combination with one or more other agents used in the treatment or suppression of disorders.

When additional active agents are used in combination with the compounds for use in the present invention, the additional active agents may generally be employed in therapeutic amounts as indicated in the Physicians' Desk Reference (PDR) 53rd Edition (1999), which is incorporated herein by reference, or such therapeutically useful amounts as would be known to one of ordinary skill in the art, or as are determined empirically for each patient.

The compounds for use in the present invention and the other therapeutically active agents can be administered at the recommended maximum clinical dosage or at lower doses. Dosage levels of the active compounds in the compositions for use in the present invention may be varied so as to obtain a desired therapeutic response depending on the route of administration, severity of the disease and the response of the patient. When administered in combination with other therapeutic agents, the therapeutic agents can be formulated as separate compositions that are given at the same time or different times, or the therapeutic agents can be given as a single composition.

In one embodiment, the purity of the preparation of the compound, such as a tocotrienol quinone preparation, is measured prior to the addition of any pharmaceutical carriers or excipients, or any additional active agents. For example, if alpha-tocotrienol quinone is prepared according to any of the methods described in International Patent Application No. PCT/US2009/062212 or U.S. patent application Ser. No. 12/606,923, the purity of the alpha-tocotrienol quinone is measured on the final product of the method selected, and prior to adding the pharmaceutical carrier(s) or excipient(s) or additional active agent(s). The purity of the desired tocotrienol quinone, or other compound, by weight, can be at least about 20%, at least about 30%, at least about 40%, at least about 50%, at least about 60%, at least about 70%, at least about 75%, at least about 80%, at least about 85%, at least about 90%, at least about 95%, at least about 96%, at least about 97%, at least about 98%, or at least about 99%, prior to the addition of any pharmaceutical carriers or excipients, or any additional active agents. These same numerical purity levels can also be used as by mole fraction, or by any other relative measurement (such as weight/volume).

In another embodiment, the purity of the preparation of the compound, such as a tocotrienol quinone preparation, is measured as a fraction of the desired tocotrienol quinone relative to the total amount of tocotrienol quinones and (if present) tocotrienols in the preparation. For example, a composition containing 100 mg of alpha-tocotrienol quinone, 50 mg of beta-tocotrienol quinone, and 50 mg of gamma-tocotrienol hydroquinone would be described as 50% alpha tocotrienol quinone by weight, irrespective of the amounts of other non-tocotrienol or non-tocotrienol quinone compounds present in the preparation. This measurement of purity would be the same whether measured before or after addition of pharmaceutical carriers or excipients, or before or after addition of any non-tocotrienol/non-tocotrienol quinone active agents. The purity of the desired tocotrienol quinone, or other compound, by weight, can be at least about 20%, at least about 30%, at least about 40%, at least about 50%, at least about 60%, at least about 70%, at least about 75%, at least about 80%, at least about 85%, at least about 90%, at least about 95%, at least about 96%, at least about 97%, at least about 98%, or at least about 99%. These same numerical purity levels can also be used as by mole fraction, or by any other relative measurement (such as weight/volume).

In another embodiment the preparation comprises 50 mg to 400 mg of alpha-tocotrienol quinone and a pharmaceutical acceptable carrier for the treatment of an individual suffering from Ataxia-Telangiectasia (A-T) or Ataxia-telangiectasia like disorder (ATLD) with one or more mutations in at least one gene located on chromosome 11q22-23 or on chromosome 11q21. In another embodiment the preparation contains 50 mg to 400 mg of alpha-tocotrienol quinone and a pharmaceutical acceptable carrier for the treatment of an individual suffering from Ataxia-Telangiectasia (A-T) or Ataxia-telangiectasia like disorder (ATLD) with one or more mutations in at least one gene located on chromosome 11q22-23 or on chromosome 11q21.

### Kits

The invention also provides articles of manufacture and kits containing materials useful for treating Ataxia-Telangiectasia (A-T) or Ataxia-telangiectasia like disorder (ATLD). The article of manufacture comprises a container with a label. Suitable containers include, for example, bottles, vials, and test tubes. The containers may be formed from a variety of materials such as glass or plastic. The container holds a compound selected from alpha-tocotrienol quinone, beta-tocotrienol quinone, gamma-tocotrienol quinone, delta-tocotrienol quinone, alpha-tocotrienol hydroquinone, beta-tocotrienol hydroquinone, gamma-tocotrienol hydroquinone, delta-tocotrienol hydroquinone and any combination of two or more of the foregoing compounds, or a composition comprising an active agent selected from alpha-tocotrienol quinone, beta-tocotrienol quinone, gamma-tocotrienol quinone, delta-tocotrienol quinone, alpha-tocotrienol hydroquinone, beta-tocotrienol hydroquinone, gamma-tocotrienol hydroquinone, delta-tocotrienol hydroquinone and any combination of two or more of the foregoing compounds. In one embodiment, the compound is alpha-tocotrienol quinone. In one embodiment, the active agent is alpha-tocotrienol quinone. The label on the container indicates that the composition is used for treating Ataxia-Telangiectasia (A-T) or Ataxia-telangiectasia like disorder (ATLD), and may also indicate directions for use in treatment.

The invention also provides kits comprising any one or more of a compound selected from alpha-tocotrienol quinone, beta-tocotrienol quinone, gamma-tocotrienol quinone, delta-tocotrienol quinone, alpha-tocotrienol hydroquinone, beta-tocotrienol hydroquinone, gamma-tocotrienol hydroquinone, delta-tocotrienol hydroquinone, and any combination of two or more of the foregoing compounds; or a composition comprising an active agent selected from alpha-tocotrienol quinone, beta-tocotrienol quinone, gamma-tocotrienol quinone, delta-tocotrienol quinone, alpha-tocotrienol hydroquinone, beta-tocotrienol hydroquinone, gamma-tocotrienol hydroquinone, delta-tocotrienol hydroquinone, and any combination of two or more of the foregoing compounds. In some embodiments, the kit of the invention comprises the container described above, which holds a compound selected from alpha-tocotrienol quinone, beta-tocotrienol quinone, gamma-tocotrienol quinone, delta-tocotrienol quinone, alpha-tocotrienol hydroquinone, beta-tocotrienol hydroquinone, gamma-tocotrienol hydroquinone, delta-tocotrienol hydroquinone and any combination of two or more of the foregoing compounds, or a composition comprising an active agent selected from alpha-tocotrienol quinone, beta-tocotrienol quinone, gamma-tocotrienol quinone, delta-tocotrienol quinone, alpha-tocotrienol hydroquinone, beta-tocotrienol hydroquinone, gamma-tocotrienol hydroquinone, delta-tocotrienol hydroquinone and any combination of two or more of the foregoing compounds. In other embodiments, the kit of the invention comprises the container described above, which holds a compound selected from alpha-tocotrienol quinone, beta-tocotrienol quinone, gamma-tocotrienol quinone, delta-tocotrienol quinone, alpha-tocotrienol hydroquinone, beta-tocotrienol hydroquinone, gamma-tocotrienol hydroquinone, delta-tocotrienol hydroquinone, or any combination of two or more of the foregoing compounds; or a composition comprising an active agent selected from alpha-tocotrienol quinone, beta-tocotrienol quinone, gamma-tocotrienol quinone, delta-tocotrienol quinone, alpha-tocotrienol hydroquinone, beta-tocotrienol hydroquinone, gamma-tocotrienol hydroquinone, delta-tocotrienol hydroquinone, and any combination of two or more of the foregoing compounds and a second container comprising a vehicle for the compound or composition, such as one or more vegetable-derived oils, such as sesame oil, and/or one or more animal-derived oils, and/or one or more fish-derived oils. In other embodiments, the kit of the invention comprises the container described above, which holds a compound selected from alpha-tocotrienol quinone, beta-tocotrienol quinone, gamma-tocotrienol quinone, delta-tocotrienol quinone, alpha-tocotrienol hydroquinone, beta-tocotrienol hydroquinone, gamma-tocotrienol hydroquinone, delta-tocotrienol hydroquinone, or any combination of two or more of the foregoing compounds; or a composition comprising an active agent selected from alpha-tocotrienol quinone, beta-tocotrienol quinone, gamma-tocotrienol quinone, delta-tocotrienol quinone, alpha-tocotrienol hydroquinone, beta-tocotrienol hydroquinone, gamma-tocotrienol hydroquinone, delta-tocotrienol hydroquinone, and any combination of two or more of the foregoing compounds where the compound or composition has been pre-mixed with a vehicle for the compound or composition, such as one or more vegetable-derived oils, such as sesame oil, and/or one or more animal-derived oils, and/or one or more fish-derived oils. The kits may further include other materials desirable from a commercial and user standpoint, including other vehicles, buffers, diluents, filters, needles, syringes, and package inserts with instructions for performing any of the methods described herein for treatment of Ataxia-Telangiectasia (A-T) or Ataxia-telangiectasia like disorder (ATLD).

In other aspects, the kits may be used for any of the methods described herein, including, for example, to treat an individual with Ataxia-Telangiectasia (A-T) or Ataxia-telangiectasia like disorder (ATLD).

### EXAMPLES

### Example 1

### Ataxia-Telangiectasia Cell Line Assay

Alpha-Tocotrienol quinone, was tested for its ability to rescue Ataxia-Telangiectasia fibroblast cells obtained from the Coriell Cell Repositories (Camden, N.J.; repository number AG04405 (AT05), when the cells were stressed by addition of L-buthionine-(S,R)-sulfoximine (BSO), as described in Jauslin et al., Hum. Mol. Genet. 11(24):3055 (2002), Jauslin et al., FASEB J. 17:1972-4 (2003), and International Patent Application WO 2004/003565.

MEM (a medium enriched in amino acids and vitamins, catalog no. 1-31F24-I) and Medium 199 (M199, catalog no. 1-21F22-I) with Earle's Balanced Salts, without phenol red, were purchased from Bioconcept. Fetal Calf Serum was obtained from PAA Laboratories. Basic fibroblast growth factor and epidermal growth factor were purchased from PeproTech. Penicillin-streptomycin-glutamine mix, L-buthionine (S,R)-sulfoximine, and insulin from bovine pancreas were purchased from Sigma. Calcein AM was purchased from Molecular Probes. Cell culture medium was made by combining 125 mL M199 EBS, 50 ml Fetal Calf Serum, 100 U/mL penicillin, 100 μg/ml streptomycin, 2 mM glutamine, 10 μg/mL insulin, 10 ng/mL EGF, and 10 ng/mL bFGF. MEM EBS was added to make the volume up to 500 mL. A 10 mM BSO solution was prepared by dissolving 444 mg BSO in 200 mL of medium with subsequent filter-sterilization. During the course of the experiments, this solution was stored at +4° C. The Ataxia telangiectasia cells were grown in 10 cm tissue culture plates. Every third day, they were split at a 1:3 ratio.

The test samples were supplied in 1.5 mL glass vials. The compounds were diluted with DMSO, ethanol or PBS to result in a 5 mM stock solution. Once dissolved, they were stored at −20° C.

Test samples were screened according to the following protocol: A culture with A-T fibroblasts was started from a 1 mL vial with approximately 500,000 cells stored in liquid nitrogen. Cells were propagated in 10 cm cell culture dishes by splitting every third day in a ratio of 1:3 until nine plates were available. Once confluent, fibroblasts were harvested. For 54 micro titer plates (96 well-MTP) a total of 14.3 million cells (passage eight) were re-suspended in 480 mL medium, corresponding to 100 μL medium with 3,000 cells/well. The remaining cells were distributed in 10 cm cell culture plates (500,000 cells/plate) for propagation. The plates were incubated overnight at 37° C. in an atmosphere with 95% humidity and 5% CO₂ to allow attachment of the cells to the culture plate.

MTP medium (243 μL) was added to a well of the microtiter plate. The test compounds were unfrozen, and 7.5 μL of a 5 mM stock solution was dissolved in the well containing 243 μL medium, resulting in a 150 μM master solution. Serial dilutions from the master solution were made. The period between the single dilution steps was kept as short as possible (generally less than 1 second).

Plates were kept overnight in the cell culture incubator. The next day, 10 μL of a 10 mM BSO solution were added to the wells, resulting in a 1 mM final BSO concentration. Forty-eight hours later, three plates were examined under a phase-contrast microscope to verify that the cells in the 0% control (wells E1-H1) were clearly dead. The medium from all plates was discarded, and the remaining liquid was removed by gently tapping the plate inversed onto a paper towel.

100 μL of PBS containing 1.2 μM Calcein AM were then added to each well. The plates were incubated for 50-70 minutes at room temperature. After that time the PBS was discarded, the plate gently tapped on a paper towel and fluorescence (excitation/emission wavelengths of 485 nm and 525 nm, respectively) was read on a Gemini fluorescence reader. Data was imported into Microsoft Excel (EXCEL is a registered trademark of Microsoft Corporation for a spreadsheet program) and used to calculate the EC₅₀ concentration for each compound.

The compound was tested three times, i.e., the experiment was performed three times, the passage number of the cells increasing by one with every repetition.

The solvents (DMSO, ethanol, PBS) neither had a detrimental effect on the viability of non-BSO treated cells nor did they have a beneficial influence on BSO-treated fibroblasts even at the highest concentration tested (1%). The compound showed no auto-fluorescence. The viability of non-BSO treated fibroblasts was set as 100%, and the viability of the BSO- and compound-treated cells was calculated as relative to this value.

Alpha-tocotrienol quinone protects the ataxia telangiectasia cells ATO5 with an ED₅₀ of 28 nM.

Similarly, the effect of alpha tocotrienol quinone on ataxia telangiectasia cells can be tested using GM01588 (AT88).)

### Example 2

### Treatment of an Ataxia-Telangiectasia (A-T) Patient

A patient with Ataxia-Telangiectasia (A-T) is treated with alpha-tocotrienol quinone. Informed consent is obtained from the child's parents in accordance with federal regulations and institutional protocol.

Alpha-tocotrienol quinone is administered to the patient mixed with sesame oil for administration. The following dosing of alpha-tocotrienol quinone is used:

### Days 0-5: 0 mg

### Days 5-12: 100 mg

Days 13 and continuing: 200 mg

While being treated with alpha tocotrienol quinone, the patient's medical team monitors the patient for any signs of improvement or signs of worsening of the disease.

### Example 3

### Treatment of an Ataxia-Telangiectasia Mutant (ATM)-Deficient Mouse

ATM-deficient mice, in 129SvEv background available from Jackson Labs (stock number 002743) are used. Mice are given chow comprising 20-100 mg/kg of test compound per day. Chow is given ad libitum.

Mice are checked for tumors and sacrificed when thymic lymphomas are detectable. The presence of tumors is confirmed histologically.

The disclosures of all publications, patents, patent applications and published patent applications referred to herein by an identifying citation are hereby incorporated herein by reference in their entirety.

Although the foregoing invention has been described in some detail by way of illustration and example for purposes of clarity of understanding, it is apparent to those skilled in the art that certain changes and modifications will be practiced. Therefore, the description and examples should not be construed as limiting the scope of the invention.

1. A method of treating Ataxia-Telangiectasia (A-T) or Ataxia-telangiectasia like disorder (ATLD) in an individual, comprising administering a therapeutically effective amount of a compound selected from the group consisting of tocotrienol quinones and tocotrienol hydroquinones, to an individual suffering from Ataxia-Telangiectasia (A-T) or Ataxia-telangiectasia like disorder (ATLD).

2. The method according to claim 1, wherein the individual is suffering from Ataxia-Telangiectasia (A-T).

3. The method according to claim 1, wherein the compound is selected from the group consisting of alpha-tocotrienol quinone, beta-tocotrienol quinone, gamma-tocotrienol quinone, and delta-tocotrienol quinone.

4. The method according to claim 1, wherein the compound is selected from the group consisting of alpha-tocotrienol hydroquinone, beta-tocotrienol hydroquinone, gamma-tocotrienol hydroquinone, and delta-tocotrienol hydroquinone.

5. The method according to claim 3, wherein the compound is alpha-tocotrienol quinone.

6. The method according to claim 1, wherein the individual suffering from Ataxia-Telangiectasia (A-T) or Ataxia-telangiectasia like disorder (ATLD) has one or more mutations in at least one gene located on chromosome 11q22-23 or on chromosome 11q21.

7. The method according to claim 1, wherein the individual is suffering from Ataxia-Telangiectasia (A-T) and has at least one mutation in at least one gene located on chromosome 11q22-23.

8. The method according to claim 1, wherein the individual is suffering from Ataxia-Telangiectasia like disorder (ATLD) and has at least one mutation in at least one gene located on chromosome 11q21.

9. The method according to claim 1, wherein the individual has one or more symptoms selected from the group consisting of: truncal ataxia; peripheral ataxia; cerebellar ataxia; chorea; atheosis; myoclonic jerks; swallowing dysfunction; tremors; dysarthria; vertical and horizontal sacchadic apraxia; telangiectasias; immunodeficiency symptoms; sinopulmonary infections; cancer; cancerous tumors; sensitivity to ionizing radiation, X rays, or gamma rays; thymic hypoplasia; hypogonadism; genomic instability; premature aging symptoms; diabetes mellitus; and progeria.

10. The method according to claim 1, wherein the individual has one or more symptoms selected from the group consisting of ataxia, telangiectasia, sinopulmonary infections, lymphomas, leukemia, breast cancer, genomic instability, and sensitivity to ionizing radiation, X rays, or gamma rays.

11. A pharmaceutical preparation containing from 50 mg to 400 mg of alpha-tocotrienol quinone and a pharmaceutically acceptable carrier for the treatment of an individual suffering from Ataxia-Telangiectasia (A-T) or Ataxia-telangiectasia like disorder (ATLD) with one or more mutations in at least one gene located on chromosome 11q22-23 or on chromosome 11q21.

12. The preparation according to claim 11, wherein the alpha-tocotrienol quinone comprises at least 50% by weight of the tocotrienols and tocotrienol quinones present in the preparation.

13. The preparation according to claim 12, wherein the alpha-tocotrienol quinone comprises at least 80% by weight of the material present in the preparation, excluding the weight of any added pharmaceutical carriers or excipients.

14. The pharmaceutical preparation according to claim 11, for use in treating Ataxia-Telangiectasia (A-T).

15. The pharmaceutical preparation according to claim 11, for use in treating an individual with Ataxia-Telangiectasia (A-T), said individual having at least one mutation on chromosome 11q22-23.

16. A unit dosage formulation of alpha-tocotrienol quinone.

17. The unit dosage formulation according to claim 16, wherein the alpha-tocotrienol quinone comprises at least 95% by weight of the tocotrienols and tocotrienol quinones present in the preparation.

18. The unit dosage formulation according to claim 17, wherein the alpha-tocotrienol quinone comprises at least 95% by weight of the material present in the preparation, excluding the weight of any pharmaceutical carriers or excipients.

19. The unit dosage formulation according to claim 16, wherein the formulation contains from 50 mg to 400 mg of alpha-tocotrienol quinone.

20. The unit dosage formulation according to claim 16, for use in treating Ataxia-Telangiectasia (A-T) or Ataxia-telangiectasia like disorder (ATLD).

21. The unit dosage formulation of claim 16, for use in treating an individual with Ataxia-Telangiectasia (A-T), said individual having at least one mutation in the gene located on chromosome 11q22-23.

22. A method of treating Ataxia-Telangiectasia (A-T) or Ataxia-telangiectasia like disorder (A-TLD) in an individual, comprising administering a therapeutically effective amount of a pharmaceutical preparation according to claim 11 to an individual suffering from Ataxia-Telangiectasia (A-T) or Ataxia-telangiectasia like disorder (A-TLD), wherein the individual has one or more symptoms selected from truncal ataxia; peripheral ataxia; cerebellar ataxia; chorea; atheosis; myoclonic jerks; swallowing dysfunction; tremors; dysarthria; vertical and horizontal sacchadic apraxia; telangiectasias; immunodeficiency symptoms; sinopulmonary infections; cancer; cancerous tumors; sensitivity to ionizing radiation, X rays, or gamma rays; thymic hypoplasia; hypogonadism; genomic instability; premature aging symptoms; diabetes mellitus; and progeria

23. The method of claim 22, wherein the individual has one or more symptoms selected from the group consisting of ataxia, telangiectasia, sinopulmonary infections, lymphomas, leukemia, breast cancer, genomic instability, and sensitivity to ionizing radiation, X rays, or gamma rays.
